(19) United States
(12) Patent Application Publication
(10) Pub. No.: US 2006/0167083 A1
(43) Pub. Date: Jul. 27, 2006

(54) Therapeutic methods and compositions involving isoflav-3-ene and isoflavan structures

(76) Inventor: Graham Edmund Kelly, Kirribilli (AU)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/510,837

(22) PCT Filed: Apr. 9, 2003
(86) PCT No.: PCT/AU03/00427

§ 371 Date: Jul. 1, 2005

(30) Foreign Application Priority Data
Apr. 9, 2002 (AU) PS 1594
Jul. 19, 2002 (AU) 2002950294
Sep. 24, 2002 (AU) 2002951607
Dec. 20, 2002 (AU) 2002953453

(51) Int. Cl. A61K 31/365; A61K 31/353

(52) U.S. Cl. 514/454

(57) ABSTRACT

Methods for the treatment of diseases associated with aberrant cell survival, aberrant cell proliferation, abnormal cell migration, abnormal angiogenesis, abnormal estrogen/androgen balance, dysfunctional or abnormal steroid genesis, degeneration including degenerative changes within blood vessel walls, inflammation, or immunological imbalances utilising isoflav-3-ene and isoflavan compounds of the general formula (II) are described. Compositions and uses involving isoflav-3-ene and isoflavan compounds are also described.

### FIELD OF THE INVENTION

The present invention relates to the regulation of cellular mechanisms by compounds based on an isoflav-3-ene or isoflavan structure and derivatives thereof. In particular, the invention relates to methods for the regulation of a range of molecular targets intimately involved in signal transduction processes in mammalian cells involving compounds based on the isoflav-3-ene or isoflavan structure, use of these compounds in the manufacture of medicaments for the regulation of cellular mechanisms, and cellular mechanism regulatory compositions comprising these compounds.

### BACKGROUND

Dehydroequol is a common name for the compound 4′,7-dihydroxyisoflav-3-ene [also known as 3-(4-hydroxyphenyl)-7-hydroxy-2H-1-benzopyran; and Haginin E], a naturally-occurring isoflav-3-ene and isoflavone metabolite. Its chemical structure is shown in Formula I:

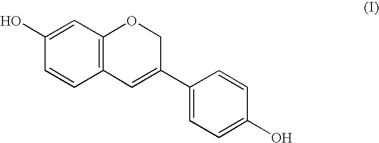

Dehydroequol was first described in 1995 by Joannou et al. [1] as a putative product of bacterial fermentation of the isoflavone, daidzein. The existence of dehydroequol was entirely speculative, with its existence not being confirmed and the compound being neither isolated nor chemically characterised.

Subsequently, dehydroequol was described as occurring naturally in the plant, Lespedeza homoloba, and termed “Haginin E” [2].

Dehydroequol was first recognised as having health benefits in animals including humans in 1997, with patent application No. WO 98/08503 entitled Therapeutic methods and compositions involving isoflavanes. The patent specification teaches that dehydroequol belongs to a family of compounds based on a primary isoflavonoid ring structure, some members of which variously display estrogenic, anti-cancer, cardiovascular and anti-inflammatory health benefits in animals. The isoflavonoid ring stricture has not been found to be an inherently bioactive structure for animals, since a large number of members of this family also display either no known biological activity in animals or display adverse, toxic biological properties.

The biochemical basis of the biological activity of dehydroequol, and for that matter other members of the chemical family cited in patent application No. WO 98/08503, remains open. Without a full understanding of biochemical activity, the range of potential health benefits of dehydroequol necessarily remains unknown, notwithstanding the proposed or known activities of other phytoestrogens and metabolites or derivatives thereof.

This application now describes new therapeutic indications for isoflav-3-ene and isoflavan components, and in particular dehydroequol, thus extending the known biological effects and health benefits of isoflav-3-enes, isoflavans and derivatives thereof. The invention is based on totally unexpected biological activities in that the applicants have surprisingly found that dehydroequol and its derivatives regulate a range of molecular targets in mammalian cells, and that these molecular targets are intimately involved in signal transduction processes that are fundamental to critical cellular processes such as cell growth, differentiation, migration and death. It can be seen therefore that these surprising biochemical effects have broad and important implications for the health of animals including humans. These and other preferred objects of the invention are described herein.

The current patent extends the biological effects and health benefits of dehydroequol and derivatives thereof.

This invention relates specifically to isoflav-3-ene and isoflavan compounds, particularly 4′,7-dihydroxyisoflav-3-ene. It is found that these compounds surprisingly regulate a wide variety of signal transduction processes within animal cells and that these signal transduction processes are involved in a wide range of functions that are vital to the survival and function of all animal cells, and that therefore these compounds have broad-ranging and important health benefits in animals including humans.

The particular benefits of this invention lie in (a) the large range of signal transduction processes targeted by the compound, (b) the fact that regulation of these various processes includes both up-regulation of some processes and down-regulation of others, and (c) that such a broad and varied effect on signal transduction processes also is accompanied by an independent effect on a range of important enzymes that are fundamental to metabolism and steroidogenesis.

It is highly unexpected that compounds of the present invention, and in particular dehydroequol, would have such broad-ranging biochemical effects with such potential for the health of animals, and particularly for the potential to prevent and treat important and common human diseases, disorders and functions.

The compounds according to the various aspects of this invention are isoflav-3-ene and isoflavan compounds of the general formula II: 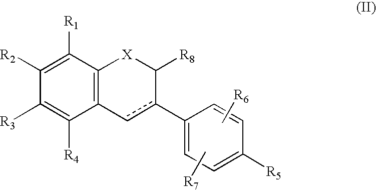
in which

R₁, R₂, R₃ and R₄ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or

R₃ and R₄ are as previously defined, and R₁ and R₂ taken together with the carbon atoms to which they are attached form a five-membered ring selected from

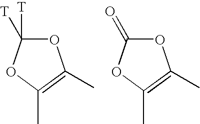

R₁ and R₄ are as previously defined, and R₂ and R₃ taken together with the carbon atoms to which they are attached form a five-membered ring selected from

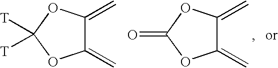

R₁ and R₂ are as previously defined, and R₃ and R₄ taken together with the carbon atoms to which they are attached form a five-membered ring selected from

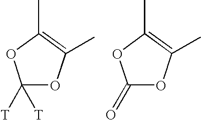

and

wherein

R₅, R₆ and R₇ are independently hydrogen, hydroxy, OR₉, OC(O)R₁₀, OS(O)R₁₀, CHO, C(O)R₁₀, COOH, CO₂R₁₀, CONR₁₁R₁₂, alkyl, haloalkyl, arylalkyl, alkenyl, alkynyl, aryl, heteroaryl, thio, alkylthio, amino, alkylamino, dialkylamino, nitro or halo,

R₈ is hydrogen, hydroxy, alkyl, aryl, amino, thio, NR₁₁R₁₂, CONR₁₁R₁₂, C(O)R₁₃ where R₁₃ is hydrogen, alkyl, aryl, arylalkyl or an amino acid, or CO₂R₁₄ where R₁₄ is hydrogen, alkyl, haloalkyl, aryl or arylalkyl,

R₉ is alkyl, haloalkyl, aryl, arylalkyl, C(O)R₁₃ where R₁₃ is as previously defined, or Si(R₁₅)₃ where each R₁₅ is independently hydrogen, alkyl or aryl,

R₁₀ is hydrogen, alkyl, haloalkyl, amino, aryl, arylalkyl, an amino acid, alkylamino or dialkylamino,

R₁₁ is hydrogen, alkyl, arylalkyl, alkenyl, aryl, an amino acid, C(O)R₁₃ where R₁₃ is as previously defined, or CO₂R₁₄ where R₁₄ is as previously defined.

R₁₂ is hydrogen, alkyl or aryl, or

R₁₁ and R₁₂ taken together with the nitrogen to which they are attached comprise pyrrolidinyl or piperidinyl,

the drawing “—” represents either a single bond or a double bond, preferably a double bond,

T is independently hydrogen, alkyl or aryl, and

X is O, NR₁₂ or S, preferably O,

including pharmaceutically acceptable salts and derivatives thereof.

In accordance with an aspect of the present invention there is provided a method for the treatment, prevention or amelioration of diseases associated with aberrant cell survival, aberrant cell proliferation, abnormal cellular migration, abnormal angiogenesis, abnormal estrogen/androgen balance, dysfunctional or abnormal steroid genesis, degeneration including degenerative changes within blood vessel walls, inflammation, and immunological imbalance, which comprises administering to a subject one or more compounds of the formula II optionally in association with a carrier and/or excipient.

In accordance with another aspect of the present invention there is provided use of compounds of the formula II in the manufacture of a medicament for the treatment, prevention or amelioration of diseases associated with aberrant cell survival, aberrant cell proliferation, abnormal cellular migration, abnormal angiogenesis, abnormal estrogen/androgen balance, dysfunctional or abnormal steroid genesis, degeneration including degenerative changes within blood vessel walls, inflammation, and immunological imbalance.

In accordance with another aspect of the present invention there is provided a method of inducing apoptosis in cells expressing abnormal prosurvival phenotype which comprises contacting said cells with one or more compounds of the formula II optionally in association with a carrier or excipient.

In accordance with another aspect of the present invention there is provided a method for inhibiting migration of cells having an abnormal cellular migration phenotype which comprises contacting said cells with a compound of the formula II optionally in association with a carrier or excipient.

In accordance with another aspect of the present invention there is provided a method for inhibiting angiogenesis in tissue expressing aberrant angiogenic phenotype which comprises contacting said tissue with a compound of the formula II optionally in association with a carrier or excipient.

In accordance with another aspect of the present invention there is provided a method for the inhibition of topoisomerase II in a mammal which method comprises the step of administering to the mammal a therapeutically effective amount of a compound of formula II or a pharmaceutically acceptable salt or derivative thereof optionally in association with a carrier or excipient.

In accordance with another aspect of the present invention there is provided a method for the treatment, prevention or amelioration of cancer in a mammal which method comprises the step of bringing a compound of formula II or a pharmaceutically acceptable salt or derivative thereof into contact with cancerous tissue in a mammal that is suffering from a tumour, such that neoplastic development in said cancerous tissue is retarded or arrested.

In a preferred embodiment, the neoplastic development is retarded or arrested by the compound of formula II stabilising a cleavable complex of DNA topoisomerase II.

In accordance with another aspect of this invention there is provided a method for the treatment, prevention or amelioration of cancer in a mammal which comprises the step of bringing a compound of the formula II or a pharmaceutically acceptable salt or derivative thereof into contact with a cancerous tissue in a mammal that is suffering from a tumour, wherein compounds of the formula II inhibit tNOX associated with said cancerous tissue, such that neoplastic development in said cancerous tissue is retarded or arrested. Preferably, compounds of the formula II, such as dehydroequol, induce apoptosis through inhibition of tNOX.

In another preferred embodiment, the compound of formula II is co-administered synergistically with a known topo II poison. In an alternative embodiment the compound of formula II is administered to a subject who has developed a tolerance or resistance to another topo II poison, or other chemotherapeutic active agent.

In accordance with another aspect of the present invention there is provided a method of inducing apoptosis in cells expressing DNA topoisomerase II which comprises contacting said cells with one or more compounds of the formula II optionally in association with a carrier or excipient.

In accordance with another aspect of the present invention there is provided a method of inhibiting DNA topoisomerase II by contacting a DNA topoisomerase cleavable complex with a compound of formula II or a pharmaceutically acceptable salt or derivative thereof to stabilise the cleavable complex.

In accordance with another aspect of the present invention there is provided use of a compound of formula II or a pharmaceutically acceptable salt or derivative thereof in the manufacture of a medicament for the treatment of cancer in a mammal.

In accordance with another aspect of the present invention there is provided use of a compound of formula II or a pharmaceutically acceptable salt or derivative thereof as a DNA topoisomerase II poison.

In accordance with another aspect of the present invention there is provided use of a compound of the formula II or a pharmaceutically acceptable salt or derivative thereof as a tNOX inhibitor. Compounds of the formula II may be used in the manufacture of a medicament for the inhibition of tNOX associated with tumour cells.

In accordance with another aspect of the present invention there is provided a pharmaceutical composition for the treatment of cancer comprising a compound of formula II or a pharmaceutically acceptable salt or derivative thereof in association with a pharmaceutically acceptable carrier and/or diluent.

In accordance with another aspect of the present invention there is provided a synergistic pharmaceutical composition comprising a compound of formula II in admixture with another chemotherapeutic active agent, preferably another topo II poison. In an embodiment, the compound of formula II is presented in a kit with another topo n poison.

In accordance with another aspect of the present invention there is provided a pharmaceutical composition comprising a compound of the formula (II) in association with one or more other pharmaceutically active agents.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

### BRIEF DESCRIPTION OF THE DRAWINGS

FIG. 1 represents an unknotting assay for determining the effect of dehydroequol on topo II catalytic activity. Substrate P4 DNA was incubated with two units of purified topo II in the absence (lane 1) or presence of 100, 80, 60, 40, 20, and 10 μg/ml dehydroequol (lanes 3-8). Lane 2, 10 μg/ml VP-16 (positive control). Topo II activity was determined by converting knotted P4 DNA (K) to the unknotted (U) form.

FIG. 2 represents a relaxation assay for determining the effect of dehydroequol on topo I catalytic activity. Substrate supercoiled pUC8 DNA was incubated with two units of purified topo I (lanes 2-8) plus camptothecin (lane 3) or varying concentrations of dehydroequol (lanes 4-8); lane 1, supercoiled pUC8 DNA, control (no topo 1); lane 2, relaxed pUC8 DNA; lane 3, 10 μg/ml camptothecin (positive control); lane 4, 100 μg/ml dehydroequol; lane 5, 80 μg/ml dehydroequol; lane 6, 60 μg/ml dehydroequol; lane 7, 40 μg/ml dehydroequol; lane 8, 20 μg/ml dehydroequol. Topo I activity was determined by converting supercoiled pUC8 DNA (SC) into its relaxed from (REL).

FIG. 3 represents an assay for determining the effect of dehydroequol on double-stranded DNA cleavage. pRYG DNA was incubated with ten units of human topo II (lanes 2-6) in the absence (lane 1) or presence of 10, 30, or 100 μg/ml dehydroequol (lanes 2-4), or 30 μg/ml genistein (lane 5), or 10 μg/ml VP-16 (lane 6). Lane 7, linear pUC8 DNA marker. Double-strand DNA breakage was determined by converting relaxed (REL) or supercoiled (SC) pRYG DNA to the linear (LIN) form.

FIG. 4 represents an assay for determining the effect of dehydroequol on single-stranded DNA cleavage. pUC8 DNA was incubated without (lane 1) or with 10 units of human topo I (lanes 2-6) under the conditions set out in the method section below. Lane 2, no inhibitor; lane 3, 10 μg/ml dehydroequol; lane 4, 100 μg/ml dehydroequol; lane 5, 10 μg/ml camptothecin; lane 6, 100 μg/ml camptothecin. Single-strand DNA breakage was determined by converting relaxed (REL) or supercoiled (SC) pUC8 DNA to the nicked form (NIC).

FIG. 5
a) Dehydroequol (□) and Genistein (▪) inhibit the proliferative potential of LNCaP cells.
b) Dehydroequol (De) inhibits growth of human xenografts in mice. De (▪) or vehicle (□) was orally administered for 5 days per week from the time of inoculation of LNCaP cells and tumour mass assessed over 58 days.
c) Dehydroequol inhibits the focus formation of Ras transformed NIH 3T3 cells compared to control wells (C) in vitro.

FIG. 6

Dehyhdroequol inhibition of endothelial cell proliferation, migration and in vitro angiogenesis.
a) EC proliferation in the presence of varying concentrations of dehydroequol (□) or DMSO vehicle (▴). Results of one experiment are given, where each group was performed in quadruplicate and is representative of 4 such experiments. Mean±SEM is given.
b) EC migration away from the wound front (white bar) over an 18 hour period in the presence of DMSO (C) or Dehydroequol (De). The results are shown of one well of duplicate wells performed in each group from one experiment representative of 2 performed.
c) In vitro angiogenesis in the presence of DMSO (C) or dehydroequol (De). One well is shown of duplicate wells performed in each group for one experiment representative of 3 performed.
d) Northern blots for mRNA of matrix metalloproteinase-2 (MMP-2) in EC treated for 18 hours with dehydroequol (De) or DMSO control (C). Top panel is MMP-2, lower panel is GAPDH.

FIG. 7
a) Expression of E selectin on endothelial cells. Group 1, unstimulated; 2, TNF stimulated for 4 hours; 3, DMSO treated for 18 h prior to TNF stimulation; 4, De treated for 1 S h prior to TNF stimulation
b) VCAM-1 expression on endothelial cells. Group 1, unstimulated; 2, TNF stimulated for 4 h or 18 h; 3, DMSO treated for 18 h prior to TNF stimulation; 4, De treated cells for 18 h prior to TNF stimulation.
One experiment of each is given, representative of 3-6 experiments performed for each.
c) IL-8 secretion from endothelial cells either unstimulated (NIL) or TNF stimulated in the presence of vehicle DMSO or dehydroequol (De). Mean±SEM of triplicate determinations in 1 of 3 experiments performed. *p<0.01

FIG. 8

Dehydroequol (De) inhibits sphingosine kinase (SK) activity of endothelial cells following stimulation with TNF (a, b), PMA (b) or IL-I (c) compared to control (C) treated wells. SK activity is given in arbitrary units. Results of between 1 and 3 experiments are shown where each group was performed in duplicate (mean±SEM). *p<0.01.

### DETAILED DESCRIPTION OF THE INVENTION

All cellular functions are under the control of a myriad of signals deriving from either distant cells (endocrine signals), neighbouring cells (paracrine signals) or from within the same cell (autocrine signals). These different signals work largely by stimulating the cell's genome (DNA) from where the appropriate cellular response is initiated. The process by which the signal is transmitted to the genome is known as signal transduction. By this we mean pathways, mostly involving different proteins, where activation of one protein catalyses the response of another protein, resulting finally in transcription of a particular gene or set of genes. Homeostasis, by which we mean the integrated functioning of cells, tissues and organs resulting in good health, is the end product of hundreds, possibly thousands, of different signals entering the body's cells on a continuous basis.

From this signalling milieu, it is possible to divide signals arbitrarily into those that are related to a ‘specialized function’, and those that are related to the fundamental ability of the cell to exist and to function. Examples of ‘specialized functions’ are pain perception by a nerve cell, production of antibodies by an immune cell, detoxification reactions by a liver cell, or formation of urine by a kidney cell. Examples of ‘fundamental functions’ are cell survival or cell death, cell proliferation, cell migration, and angiogenesis. It can be seen that the key to regulating whether or not a cell is able to perform ‘specialized functions’ is regulation of the cell's ‘fundamental functions’.

In a surprising and major discovery, the applicants have found that compounds of the formula II, particularly dehydroequol, regulate many of the ‘fundamental functions’ of the cell. This discovery is surprising (a) because it has not been considered possible up until now for a single compound to have such comprehensive actions against so many targets that are involved in the fundamental regulatory mechanisms in a cell, and (b) the mode of action by which dehydroequol regulates these molecular targets is entirely novel, modifying the action of these targets only when they are dysfunctional. Further, it is a major discovery, because a compound that has such a biological effect has clear and important implications to the ability of cells to function across their full spectrum of activity, and in turn this has substantial implications to the general health of animals.

In the description which follows, particular reference is made to dehydroequol. However, this description is to be understood to apply to other compounds of the formula II.

The following are some examples of the ‘fundamental functions’ the inventors have surprisingly found are regulated by dehydroequol, and other compounds of the formula II.

1. Cell Survival/Death

In order to continue to function, including the ability to respond to specialized functions, cells need to be continuously activating pro-survival signal transduction mechanisms. Pro-survival mechanisms act at two main levels—those that actively promote survival and those that actively suppress cell death (apoptosis).

Pro-survival mechanisms involve a number of different signal transduction processes that ultimately cause transcription of certain genes whose end-products promote cell survival. These different processes involve, but are limited to, such molecular targets as MEK, ERK, and NFκB. Dehydroequol has been found to operate across a range of these processes. One in particular by way of example is the enzyme, sphingosine kinase. Sphingosine kinase phosphorylates the substrate, sphingosine to sphingosine-1-phosphate. Sphingosine-1-phosphate is an important stimulator of pro-survival mechanisms and is over-expressed in a range of disease states characterized by increased longevity of cells. Dehydroequol down-regulates sphingosine kinase activity.

Apoptosis can be achieved by a number of mechanisms as follows.
(a) One such mechanism involves receptors known as ‘death receptors’. These include receptors such as Fas/Mort, TGF and TNRF. Activation of receptors normally is suppressed through the production of blocking proteins such as C-flip. Dehydroequol has been found to block the production of C-flip, in so doing, promoting the death of cells.
(b) Another mechanism involves the activation of proteolytic enzymes known as caspases. Once activated, these enzymes autolyse the cell. Dehydroequol has been found to up-regulate the activity of caspases.
(c) Another mechanism involves disruption of mitochondria leading to the production of various pro-death factors. Dehydroequol has been found to promote such disruption through a direct and novel effect on the mitochondria.

It can be seen from the above description, that dehydroequol is able to induce cell death in a comprehensive manner via a number-of different pathways. The ability of a single compound to have such broad and complementary effects is novel. But of considerable surprise is the finding that dehydroequol exerts such pro-death effects in abnormal cells only.

That is, in normal healthy cells, dehydroequol has no discernible effect on these regulatory processes. Cells that display abnormal activity of these regulatory processes include but are not limited to cells involved in such disease states as cancer, cardiovascular disease, autoimmune diseases, and diseases with immunological, inflammatory or hyperproliferative components.

2. Cell Proliferation

The ability to divide in response to growth signals is another fundamental function required by normal, healthy cells. Sphingosine-1-phosphate appears to play a key role in facilitating the ability of cells to divide. The act of cell division involves a number of different enzymes as follows:
(a) the activation of topoisomerases (I and II) whose task it is to organize DNA prior to mitosis;
(b) the activation of cyclin dependent kinases (CDKs) whose task is it to move the genome through the different stages of mitosis;
(c) inactivation of cyclin dependent kinase inhibitors (CDKIs) whose task it is to inhibit mitosis through suppression of CDKs.

Dehydroequol surprisingly inhibits all 3 above components, viz. topoisomerase II, CKDs and CDKIs. While various drugs have been described that inhibit each of these components separately, the concept that a single drug might inhibit all three distinctive enzyme systems is novel and surprising.

Contributing to the novelty and surprise is the fact that dehydroequol only inhibits these enzyme systems in cells that are behaving abnormally, particularly cells expressing abnormal prosurviving phenotype or aberrant cell proliferation.

3. Cell Migration

It is well understood that the ability of a cell to migrate and to interact with its neighbouring cells is fundamental to health and disease. Sphingosine kinase and matrix-metalloproteases are key regulators of this important cell function. Dehydroequol uniquely down-regulates both of these enzyme systems, thus diminishing the ability of cells in a diseased state to migrate.

4. Angiogenesis

The ability to form new blood vessels is well known to be a key event underlying many disease states associated with hyperplasia. Sphingosine kinase is a key facilitator of this event. Dehydroequol by down-regulating this enzyme, selectively impairs angiogenesis when it occurs in association with disease, and not in healthy tissues.

These broad-ranging effects of dehydroequol on signal transduction mechanisms are complemented surprisingly by inhibitory effects on a wide range of enzymes, such enzymes not normally being regarded as part of signal transduction processes, but of the physiology of the body in more general terms. These effects also include the following:

5. Steroidogenesis

Dehydroequol inhibits a number of enzymes involved in steroidogenesis. These include but are not limited to steroid dehydrogenase, 5-α-reductase and aromatase. People skilled in the art would recognize that such effects would have significant impact on the production of steroid hormones including androgens, estrogens and corticosteroids. Such effects would be regarded as someone skilled in the art in having impact on the normal function of the male and female reproductive tissues including the breast, ovary, uterus, endometrium, cervix, vagina, prostate and penis.

In summary, the inventors have surprisingly found that dehydroequol regulates a unique collection of enzymes involved in both general metabolism and physiological function, and in signal transduction pathways that play pivotal roles in cell survival, cell growth, cell differentiation, and cell response to inflammation and immune modulators. Through regulation of this group of enzymes the compounds of the invention have the capacity to (a) to prevent or to treat many forms of disease irrespective of the cause or pathogenesis of that disease, and (b) influence the full range of biological activities of the body's tissues and the way in which disease, age, environmental influences and other drugs influence those activities.

Moreover, it is highly surprising and novel to find that a compound that can cause a human breast cancer cell to undergo apoptosis and die, also can have such diverse effects as antagonising hypertension, redressing the immunological and inflammatory imbalance underlying inflammatory bowel disease, reversing Type I diabetes, and reversing male pattern baldness. There is no known causative or pathogenic link between any or all of these disorders making it entirely unexpected that dehydroequol should display such health benefits.

Without prejudicing the full importance of dehydroequol across the broad range of biological activities in the body, it can readily be seen that this compound would have particular relevance in the prevention and treatment of various disease states and disorders as follows.

A. Diseases and Disorders Associated with Abnormal Response to Growth Signals, Abnormal Cellular Proliferation, Dysfunctional Apoptosis, and Abnormal Migration Patterns (Metastasis)

These include:
1. all forms of cancer (pre-malignant, benign and malignant) in all tissues of the body. In this regard, the compounds may be used as the sole form of anti-cancer therapy or in combination with other forms of anti-cancer therapy including but not limited to radiotherapy and chemotherapy;
2. papulonodular skin lesions including but not limited to sarcoidosis, angiosarcoma, Kaposi's sarcoma, Fabry's Disease
3. papulosquamous skin lesions including but not limited to psoriasis, Bowen's Disease, and Reiter's Disease;
4. proliferative disorders of bone marrow including but not limited to megaloblastic disease, myelodysplastic syndromes, polycythemia vera, thrombocytosis and myelofibrosis;
5. hyperplastic diseases of the reproductive tract including but not limited to benign prostatic hyperplasia, endometriosis, uterine fibroids, and polycystic ovarian disease.
   B. Diseases and Disorders Associated with Abnormal Angiogenesis

These include:
1. diseases and disorders associated with abnormal angiogenesis affecting any tissue within the body including but not limited to metastatic cancers, psoriasis, hemangiomas and telangiectasia.
   C. Diseases and Disorders Associated with Abnormal Inflammatory/Immunological Responses

These include:
1. diseases and disorders associated with inflammatory reactions of an abnormal or prolonged nature in any of the body's tissues including but not limited to rheumatoid arthritis, tendonitis, inflammatory bowel disease, ulcerative colitis, Crohn's Disease, sclerosing cholangitis;
2. diseases and disorders associated with degenerative changes within the walls of blood vessels including but not limited to the syndrome known commonly as cardiovascular disease (embracing the diseases atherosclerosis, atheroma, coronary artery disease, stroke, myocardial infarction, post-angioplasty restenosis, hypertensive vascular disease, malignant hypertension, thromboangiitis obliterans, fibromuscular dysplasia);
3. diseases and disorders associated with abnormal immunological responses including but limited to dermatomyositis and scleroderma.
4. immunological imbalance including immune deficiency associated with H.I.V. or other viral infective agents or bacterial infective agents, and immune deficiency related to immaturity or aging.
   D. Diseases and Disorders Associated with Decreased Cellular Function Including Depressed Response to Growth Signals and Increased Rates of Cell Death

These include:
1. actinic damage characterized by degenerative changes in the skin including but not limited to solar keratosis, photosensitivity diseases, and wrinkling;
2. autoimmune disease characterized by abnormal immunological responses including but not limited to multiple sclerosis, Type I diabetes, systemic lupus erythematosis, and biliary cirrhosis;
3. neurodegenerative diseases and disorders characterized by degenerative changes in the structure of the neurological system including but not limited to Parkinson's Disease, Alzheimer's Disease, muscular dystrophy, Lou-Gehrig Disease, motorneurone disease;
4. diseases and disorders associated with degenerative changes within the eye including but not limited to cataracts, macular degeneration, retinal atrophy.
   E. Diseases and Disorders Associated with Dysfunctional or Abnormal Steroidogenesis and Function of Reproductive Hormones

These include:
1. conditions in women associated with abnormal estrogen/androgen balance including but not limited to cyclical mastalgia, acne, dysmenorrhoea, uterine fibroids, endometriosis, ovarian cysts, premenstrual syndrome, acute menopause symptoms, osteoporosis, senile dementia, infertility;
2. conditions in men associated with abnormal estrogen/androgen balance including but not limited to benign prostatic hypertrophy, infertility, gynecomastia, alopecia hereditaria and various other forms of baldness.

In an important field of study the inventors have been investigating cell proliferation and the factors affecting the ability of cells to divide by mitosis. One of the important classes of enzymes involved in mitosis is the topoisomerases, whose task it is to organise DNA prior to mitosis.

More specifically DNA topoisomerases constitute a family of conserved essential enzymes that resolve topological problems during DNA replication transcription and recombination. The mammalian type-I enzyme (or topo I) is an ATP-independent DNA single-strand endonuclease and ligase that functions mainly during transcription. The mammalian type II enzyme (or topo II) is represented by two isoforms (α and β) that are ATP-dependent DNA double-stranded endonucleases and ligases. Topo IIα is a major component of the chromosomal matrix that decatenates double-stranded DNA during replication. The expression of topo IIα is cell cycle-regulated and proliferation-dependent, whereas the expression of topo I and topo IIβ are relatively constant throughout the cell cycle and independent of proliferation [3].

Inhibition of topo II may generally take place by either (a) stabilising a transient reaction intermediate between the topo II enzymes and DNA (called the cleavable complex) or (b) hindering its formation [4]. Topo II inhibitors that stabilise the cleavable complex are named topo II poisons and are represented by antitumour drugs such as VP-16 (etoposide) and doxorubicin. Topo II inhibitors that do not stabilise the cleavable complex are named catalytic inhibitors and are represented by agents such as aclarubicin and merbarone that may or may not find applications as cancer therapeutics.

Topo II poisons are cytotoxic due to the production of double-strand breakage that may escape the repair process. Tumour cells that contain higher levels of topo II are more susceptible to the cytotoxic effects of topo II poisons than normal, non-dividing cells, which generally contain very low topo II levels [5-8].

Previously, the soy isoflavone genistein has been identified as a topo II poison, as it inhibits the catalytic activity of topo II and stabilises the cleavable complex [4, 9-13]. In this regard, genistein can act as an antitumour drug when introduced at high concentration [14], but also, like many other antitumour drugs, it is thought that it contributes to the promotion of human leukemias [15].

Accordingly, there is a continual need to find new or improved compounds and compositions which exhibit physiological properties important to the health and well-being of mammals, particularly humans, and to find new methods which exploit these properties for the treatment, amelioration and prophylaxis of disease.

The present inventors have made the surprising discovery that dehydroequol is a potent topo II poison binding to a novel site on the topoisomerase/DNA cleavable complex. This provides for the use of dehydroequol and derivatives thereof both in new applications in cancer chemotherapy as well as in enhancing the antitumour effects of known topo II poisons.

It is highly unexpected that the isoflav-3-ene and isoflavan compounds of the present invention, and in particular dehydroequol, would inhibit DNA topoisomerase II with such specificity and utility via novel binding configurations with the cleavable complex so as to highlight their potential in the prevention and treatment of related mammalian diseases, disorders and functions.

Applicants have now shown for the first time that dehydroequol is a topo II-specific poison. Dehydroequol was found not to inhibit the topo I catalytic activity nor was it found to trap the topo I-cleavable complex. The specificity of dehydroequol towards topo II places it in the same category as the most widely prescribed antineoplastic drugs that target topo II [16]. Topo I levels are relatively similar between normal and tumour cells. Contrary to that, topo II levels are much higher in rapidly dividing tumour cells. Consequently, agents that act as topo II poisons direct their cytotoxic effects mainly against tumour cells, while those that act as both topo I and II poisons may also be cytotoxic to normal cells. This observation is consistent with the observed low toxicity of dehydroequol in normal healthy tissues found by the applicants.

The ability of dehydroequol to promote topo II-mediated DNA cleavage in vitro by stabilising the cleavable complex is comparable to that of other topo II poisons that are currently used in cancer chemotherapy. One such drug is VP-16, used in the treatment of small cell lung carcinoma, which yields remissions in 70% of patients and is considered to be a “pure” topo II poison [17].

The applicants have surprisingly found that dehydroequol produces detectable topo II-mediated linear plasmid DNA at a concentration of 20 μg/ml. This is lower than the concentration of genistein (30 μg/ml) that produced comparable DNA cleavage. The effect of dehydroequol was similar to that of VP-16.

Topo II poisons, including VM-26, VP-16, doxorubicin, amsacrine, and several dietary bioflavonoids, represent a class of topo II inhibitors that convert a normal enzyme (topo II) into a cellular poison. The ternary complexes, formed between topo II, DNA, and the drug, are initially reversible by DNA religation or DNA repair [4]. Cellular processing of the accumulating ternary complexes activates an irreversible step that leads to protein-associated DNA fragments 300-600 kb in size [18]. Following this irreversible step, caspase 3 becomes activated, which produces endonucleolytic DNA cleavage characteristic of apoptosis. Thus, following DNA replication or transcription, these topo II poisons convert cleavable complexes into lethal lesions [17, 19]. The sensitivity of tumour cells to topo II inhibitors is strongly associated with intranuclear topo II levels [5, 7, 8]. Since rapidly dividing lung cancer, breast cancer, ovarian cancer, and malignant lymphoma cells generally express much higher levels of topo II than normal non-dividing cells, the former are more susceptible to the deleterious effects of topo II poisons. Furthermore, reduced topo II activity has been associated with cell differentiation [4, 9]. Based on the effects of dehydroequol on topo II activity, this agent is expected to induce tumour cell differentiation and activate the apoptotic pathway. These biological effects of dehydroequol are consistent with its ability to inhibit topo II and produce double-strand DNA breaks.

The catalytic cycle of topo II can be divided into six discrete steps. These are: 1) binding of topo II to DNA, 2) double-stranded DNA cleavage, 3) double-stranded passage through the break, 4) religation of the cleaved DNA, 5) ATP hydrolysis, and 6) enzyme turnover [20].

The clinical applications of topo II poisons depend on the exact steps of the catalytic cycle that are inhibited It has been established by the applicants in this patent that dehydroequol traps the cleavable complex, however it is not clear whether this is accomplished by enhancing the cleavage step, by inhibiting the religation step or by some combination of both steps. That is it is not yet clear whether dehydroequol binds to topo II, the DNA, or the topo II/DNA complex.

Topo II poisons such as daunorubicin, doxorubicin, amsacrine, ellipticine, and mitoxantrone are DNA intercalators [21]. Other topo II poisons such as VP-16, VM-26, clerocidin, and salvicine do not intercalate to the DNA [21, 22]. The clinical applications of dehydroequol and its derivatives includes synergistic compositions with other chemotherapeutic agents and its use in the treatment of patients who have developed a resistance to presently administered chemotherapeutic agents. That is, where dehydroequol binds to a different topo II site than known topo II poisons, such as VP-16, it finds application in the treatment of carcinomas expressing mutant forms of topo II that do not bind to the known topo II poison, therefore escaping its cytotoxic effects.

NAD(P)H oxidase (NOX) proteins are described for example in Morré et al (2002) Biochemistry, Vol. 41 No. 40, pages 11941-11945 [24]. Such NADH oxidases at the external surface of animal cells exhibit stable and recurring patterns of oscillations with clock-related, entrainable, and temperature compensated periods of 24 minutes. These proteins are characterised by the property, unprecedented in the biochemical literature, of having two distinct biochemical activities, hydroquinone (NAD(P)H) oxidation and protein disulphide-thiol interchange that alternate (Morré et al supra). Such proteins may be referred to as ECTO-NOX proteins because of their cell surface location (Morré, D. J. (1995) Biochim. Biophys. Acta. 1240, 201-208 [25]). The constitutive ECTO-NOX, designated CNOX, is hormone responsive and refractory to quinone-site inhibitors. NOX associated with tumour cells (tNOX) is unregulated, refractory to hormones and growth factors, and responds to inhibitors (Morrê, D. J. (1998) in Plasma Membrane Redox Systems and Their Role in Biological Stress and Disease (Asard et al editors), pp 121-156,

Kluwer Academic Publishers, Dordrecht, the Netherlands [26]). CNOX proteins are widely distributed and exhibit activity oscillations with a period length of 24 minutes. tNOX proteins on the other hand are cancer cell specific and exhibit oscillations with a period length of about 22 minutes, that is 2 minutes shorter than those of CNOX (Wang et al (2001) Biochim. Biophys. Acta. 1539, 192-204 [27]).

The disulphide-thiol interchange activity of NOX proteins drives cell enlargement, which when inhibited results in apoptosis. The inventors have shown that compounds of the formula II, such as dehydroequol, are potent inhibitors of the disulphide-thiol interchange of tNOX by blocking tNOX and thus cell enlargement. The resultant small cells, being unable to divide, undergo G₁ cell cycle arrest, which leads to apoptosis. Compounds of the formula (II), such as dehydroequol, selectively inhibit tNOX, whereas tNOX is not so inhibited. This selectivity is believed to be of particular therapeutic significance in the treatment of cancers including solid tumours and metastasis.

Compounds of the formula (II), including dehydroequol, have been found by the applicants to inhibit matrix degrading enzymes such as metalloproteases, particularly matrix-metalloproteases. Angiogenesis associated with disease states such as tumour growth and inflammation is dependent on the synthesis and secretion of matrix-metalloproteases. Accordingly, compounds of the present invention may be used to inhibit matrix metalloproteases in the treatment of diseases associated with angiogenesis and inflammation.

The isoflav-3-ene and isoflavan compounds of the invention are set out in general formula II above. Preferred compounds of the invention are of the general formula III:

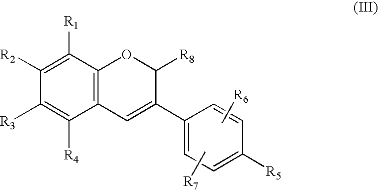

in which

R₁, R₂, R₃, R₄, R₅, R₆, R₇ and R₈ are as defined above;

more preferably

R₁, R₂, R₃, R₄, R₅, R₆ and R₇ are independently hydrogen, hydroxy, OR₉, OC(O)R₁₀, C(O)R₁₀, COOH, CO₂R₁₀, alkyl, haloalkyl, arylalkyl, aryl, thio, alkylthio, amino, alkylamino, dialkylamino, nitro or halo,

R₈ is hydrogen, hydroxy, alkyl, aryl, COR₁₃ where R₁₃ is as previously defined, or CO₂R₁₄ where R₁₄ is as previously defined,

R₉ is alkyl, haloalkyl, arylalkyl, or C(O)R₁₃ where R₁₃ is as previously defined, and

R₁₀ is hydrogen, alkyl, amino, aryl, an amino acid, alkylamino or dialkylamino,

more preferably

R₂ is hydroxy, OR₉, OC(O)R₁₀ or halo,

R₁, R₃, R₄, R₅, R₆ and R₇ are independently hydrogen, hydroxy, OR₉, OC(O)R₁₀, C(O)R₁₀, COOH, CO₂R₁₀, alkyl, haloalkyl, or halo,

R₈ is hydrogen,

R₉ is alkyl, arylalkyl or C(O)R₁₃ where R₁₃ is as previously defined, and

R₁₀ is hydrogen or alkyl,

and more preferably

R₂ is hydroxy, methoxy, benzyloxy, acetyloxy or chloro,

R₁, R₃, R₄, R₅, R₆ and R₇ are independently hydrogen, hydroxy, methoxy, benzyloxy, acetyloxy, methyl, trifluoromethyl or chloro, and

R₈ is hydrogen,

including pharmaceutically acceptable salts and derivatives thereof.

Still further particularly preferred compounds of the present invention are selected from the isoflav-3-ene compounds 1 to 40:

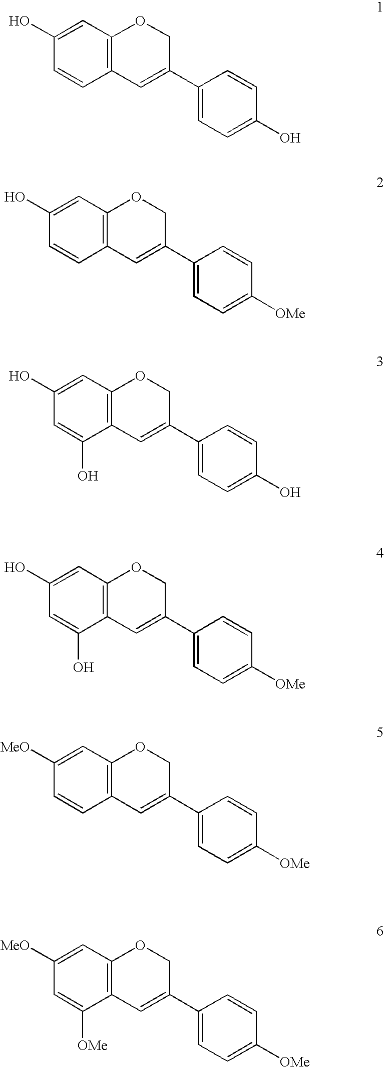

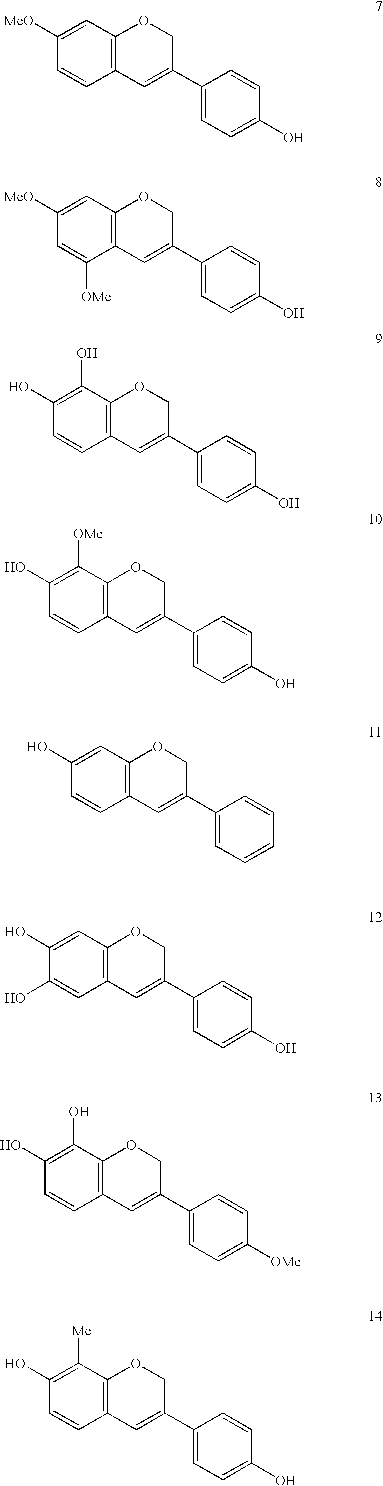

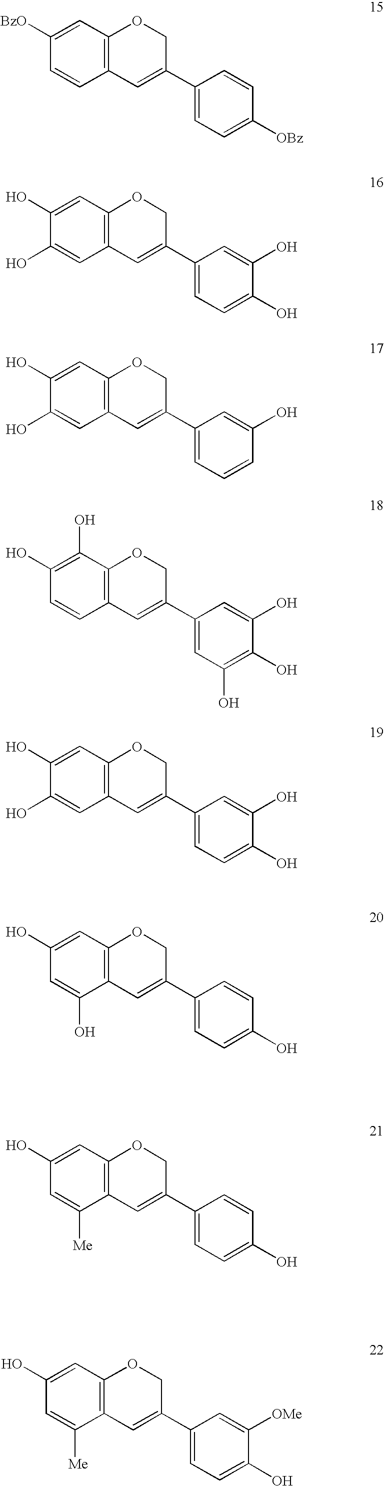

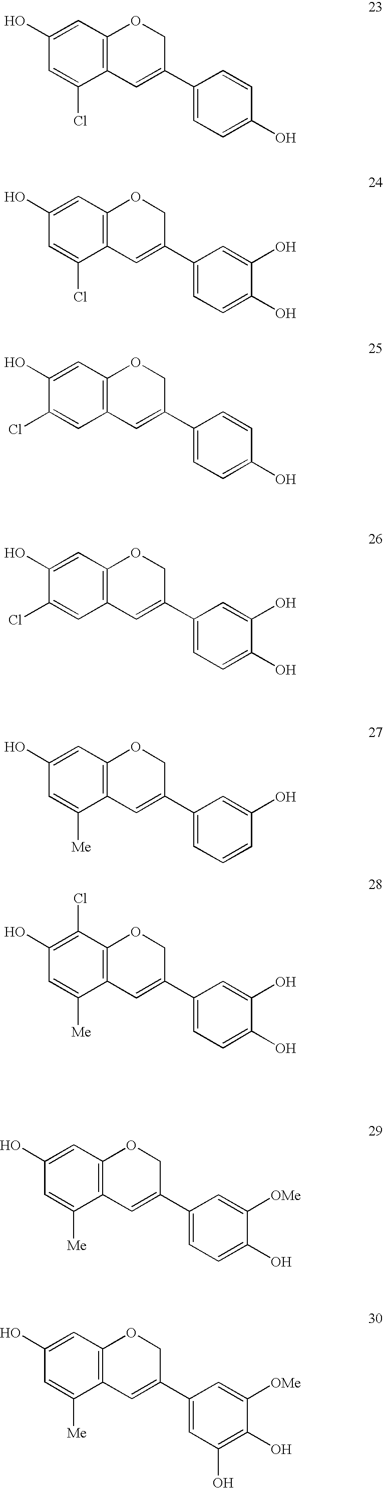

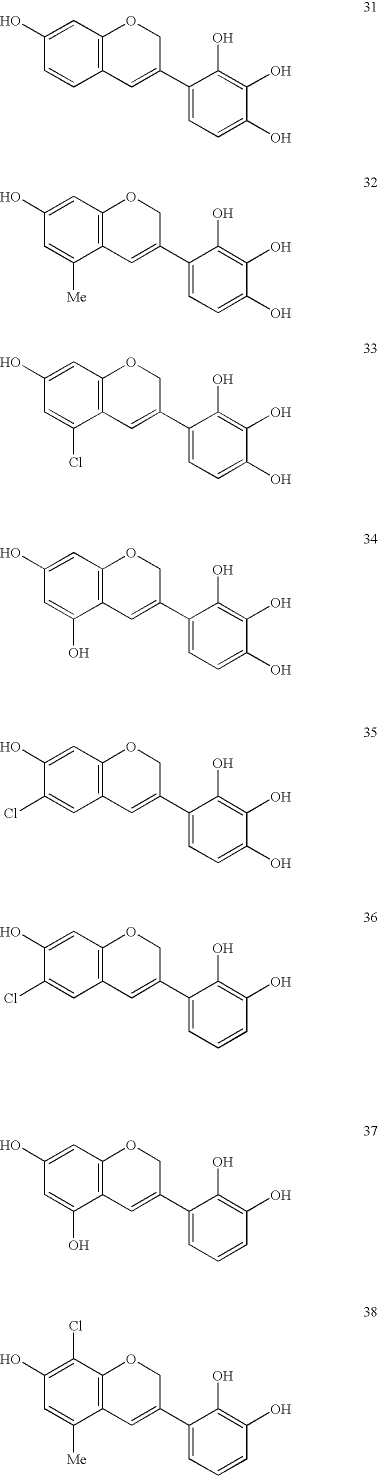

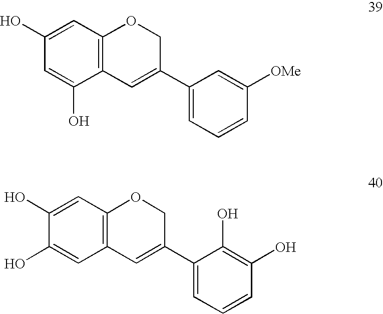

In a most preferred embodiment of the invention the compound is compound 1, dehydroequol.

Further preferred compounds of the invention are of the general formula IV:

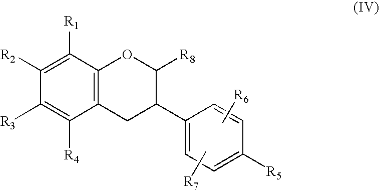

in which

R₁, R₂, R₃, R₄, R₅, R₆, R₇ and R₈ are as defined above.

In a more particularly preferred embodiment, the compounds of the invention are those isoflavan compounds of general formula IV which directly correspond to their isoflav-3-ene counterparts described above. In these compounds numbered 41 to 80, the 3-ene pyran-ring double bond of compounds 1 to 40 respectively is now a single bond.

The preferred compounds of the present invention also include all derivatives and prodrugs with physiologically cleavable leaving groups that can be cleaved in vivo from the isoflavene, isoflavan or derivative molecule to which it is attached. The leaving groups include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted isoflavenes and derivatives thereof are readily cleavable to the corresponding hydroxy substituted compounds. In addition, the protection of functional groups on the isoflavene compounds and derivatives of the present invention can be carried out by well established methods in the art, for example as described in T. W. Greene, Protective Groups in Organic Synthesis, John Wiley & Sons, New York, 1981.

Reference to a compound of the invention includes reference to one or more of the compounds. Reference to the use of a compound of the invention includes reference to the use of that compound by itself, in association with an excipient and/or diluent, and/or in association with one or more further active agents.

The term “alkyl” is taken to include straight chain, branched chain and cyclic (in the case of 5 carbons or greater) saturated alkyl groups of 1 to 10 carbon atoms, preferably from 1 to 6 carbon atoms, such as methyl, ethyl, propyl, isopropyl, butyl, isobutyl, secbutyl, tertiary butyl, pentyl, cyclopentyl, and the like. The alkyl group is more preferably methyl, ethyl, propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkenyl” is taken to include straight chain, branched chain and cyclic (in the case of 5 carbons or greater) hydrocarbons of 2 to 10 carbon atoms, preferably 2 to 6 carbon atoms, with at lease one double bond such as ethenyl, 1-propenyl, 2-propenyl, 1-butenyl, 2-butenyl, 2-methyl-1-peopenyl, 2-methyl-2-propenyl, and the like. The alkenyl group is more preferably ethenyl, 1-propenyl or 2-propenyl. The alkenyl groups may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-acyloxy, formyloxy, C₁-C₄-alkyl-carbonyloxy, C₁-C₄-alkylthio, C₃-C₆-cycloalkyl or phenyl.

The term “alkynyl” is taken to include both straight chain and branched chain hydrocarbons of 2 to 10 carbon atoms, preferably 2 to 6 carbon atoms, with at least one triple bond such as ethynyl, 1-propynyl, 2-propynyl, 1-butynyl, 2-butynyl, and the like. The alkynyl group is more preferably ethynyl, 1-propynyl or 2-propynyl. The alkynyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “heteroaryl” is taken to include five-membered and six-membered rings which include at least one oxygen, sulfur or nitrogen in the ring, which rings may be optionally fused to other aryl or heteroaryl rings including but not limited to furyl, pyridyl, pyrimidyl, thienyl, imidazolyl, tetrazolyl, pyrazinyl, benzofuranyl, benzothiophenyl, quinolyl, isopuinolyl, purinyl, morpholinyl, oxazolyl, thiazolyl, pyrrolyl, xanthinyl, purine, thymine, cytosine, uracil, and isoxazolyl. The heteroaromatic group can be optionally substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl. The heteroaromatic can be partially or totally hydrogenated as desired.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The term “pharmaceutically acceptable salt” used herein refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient is capable of providing directly or indirectly, the parent compound or metabolite, or that exhibits activity itself.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The amount of one or more compounds of formula II which is required in a therapeutic treatment according to the invention will depend upon a number of factors, which include the specific application, the nature of the particular compound used, the condition being treated, the mode of administration and the condition of the patient. Compounds of formula II may be administered in a manner and amount as is conventionally practised. See, for example, Goodman and Gilman, et al. (1995) The Pharmacological Basis of Therapeutics 8th Edition. The specific dosage utilised will depend upon the condition being treated, the state of the subject, the route of administration and other well known factors as indicated above. In general, a daily dose per patient may be in the range of 0.1 mg to 5 g; typically from 0.5 mg to 1 g; preferably from 50 mg to 200 mg. The length of dosing may range from a single dose given once every day or two, to twice or thrice daily doses given over the course of from a week to many months to many years as required, depending on the severity of the condition to be treated or alleviated. It will be further understood that for any particular subject, specific dosage regimens should be adjust over time according to the individual need and the professional judgment of the person administering or supervising the administration of the compositions.

Relatively short term treatments with the active compounds can be used to cause stabilisation or shrinkage of coronary artery disease lesions that cannot be treated either by angioplasty or surgery. Longer term treatments can be employed to prevent the development of advanced lesions in high-risk patients.

The production of pharmaceutical compositions for the treatment of the therapeutic indications herein described are typically prepared by admixture of the compounds of the invention (for convenience hereafter referred to as the “active compounds”) with one or more pharmaceutically or veterinary acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain up to 100% by weight of the active compound, preferably from 0.5% to 59% by weight of the active compound. One or more active compounds may be incorporated in the formulations of the invention, which may be prepared by any of the well known techniques of pharmacy consisting essentially of admixing the components, optionally including one or more accessory ingredients. The preferred concentration of active compound in the drug composition will depend on absorption, distribution, inactivation, and excretion rates of the drug as well as other factors known to those of skill in the art.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and are administered at a rate of 0.1 ml/minute/kg.

Formulations suitable for rectal administration are preferably presented as unit dose suppositories. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 5% w/w, more particularly from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound.

Formulations suitable for transdermal administration may also be delivered by iontophoresis (see, for example, Panchagnula R, et al., 2000 Transdermal iontophoresis revisited Current Opinion Chemical Biology Vol 4, Issue 4, pp 468-473) and typically take the form of an optionally buffered aqueous solution of the active compound. Suitable formulations comprise citrate or Bis/Tris buffer (pH 6) or ethanol/water and contain from 0.1 M to 0.2 M active ingredient.

Formulations suitable for inhalation may be delivered as a spray composition in the form of a solution, suspension or emulsion. The inhalation spray composition may further comprise a pharmaceutically acceptable propellant such as carbon dioxide or nitrous oxide.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Therapeutic methods, uses and compositions may be for administration to humans or animals, including mammals such as companion and domestic animals (such as dogs and cats) and livestock animals (such as cattle, sheep, pigs and goats), birds (such as chickens, turkeys, ducks) and the like.

The active compound or pharmaceutically acceptable derivatives prodrugs or salts thereof can also be co-administered with other pharmaceutically active materials that do not impair the desired action, or with materials that supplement the desired action, such as antibiotics, antifungals, antinflammatories, or antiviral compounds. The active agent can comprise two or more isoflavones or derivatives thereof in combination or synergistic mixture. The active compounds can also be administered with lipid lowering agents such as probucol and nicotinic acid; platelet aggregation inhibitors such as aspirin; antithrombotic agents such as coumadin; calcium channel blockers such as verapamil, diltiazem, and nifedipine; angiotensin converting enzyme (ACE) inhibitors such as captopril and enalapril, and β-blockers such as propanolol, terbutalol, and labetalol. The compounds can also be administered in combination with nonsteriodal antiinflammatories such as ibuprofen, indomethacin, aspirin, fenoprofen, mefenamic acid, flufenamic acid and sulindac. The compounds can also be administered with corticosteroids.

In an important aspect of the present invention, a compound of the formula II is compounded with another cytotoxin or chemotherapeutic agent, and, in particular, agents which also stabilise the cleavable complex or hinder its formation. Preferred agents are VP-16 (etoposide) and doxorubicin, however this aspect of the invention is not necessarily to be limited to these two known agents. These compounds are thought to exhibit synergistic activity against cancerous cells and tumours. Without wishing to be limited to theory, the synergism is thought to be based on the ability of the compounds of the present invention to bind to novel topo II sites. Thus the compounds of formula II find application in cancer therapy where cells express mutant forms of topo II which show resistance to existing topo II poisons.

The co-administration may be simultaneous or sequential. Simultaneous administration may be effected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

Synthesis

Synthesis of the compounds of the formula II can be achieved by a number of routes. Particular reference is made to International patent application WO00/49009, and references cited therein, which are incorporated herein in their entirety by reference. The International application describes improved methods of preparing isoflavenes from simple, readily available starting materials. A convenient starting material is daidzein which is readily obtained by established routes.

In a general synthesis, daidzein is protected as its di-acetate, and then dehydrogenated to tetrahydrodaiczein diacetate in near quantitative yield. This general synthetic method allows access to clean and near quantitative yields of other isoflavan-4-ol compounds by hydrogenation of the corresponding isoflavone.

Dehydration of the isoflavan-4-ol with standard reagents such as strong acids or P₂O₅ and the like leads to the unsaturated isoflav-3-enes of the invention. The dehydration reactions can be carried out on the hydrogenation products directly, or deprotected derivatives thereof.

Synthesis of dehydroequol (I) was achieved by removal of the protecting acetoxy groups under mild conditions. Other isoflav-3-ene derivatives may be prepared by similar methods.

Isoflavans for use in the present invention can be readily prepared by the hydrogenation of isoflav-3-enes or isoflavones or other known procedures in the art.

The isoflav-3-enes for use in the present invention may also be synthesised from isoflavones derived from any number of sources readily identifiable to a person skilled in the art. Preferably, they are obtained in the form of concentrates or extracts from plant sources. Again, those skilled in the art will readily be able to identify suitable plant species, however, for example, plants of particular use in the invention include leguminous plants. More preferably, the isoflavone extract is obtained from chickpea, lentils, beans, red clover or subterranean clover species and the like.

The present invention will now be described with reference to the following non-limiting examples.

### EXAMPLE 1

Materials and Methods

Dehydroequol was evaluated as a potential inhibitor of topoisomerases, by using the relaxation and nicking assays that can identify topo I inhibitors, and the unknotting and DNA cleavage assays that can identify topo II inhibitors. Dehydroequol inhibited the catalytic activity of topo II in a dose-dependent manner and it stabilised the topo II-mediated cleavable complex, demonstrating that this agent is a topo II poison. Dehydroequol's topo II inhibitory effects were comparable to those of other antitumour agents such as VP-16 and were stronger than those of genistein. Dehydroequol did not inhibit topo I catalytic activity nor did it stabilise the topo I-mediated cleavable complex. These results demonstrate that dehydroequol is a topo II-specific poison and support its application in cancer chemotherapy.

Inhibition of the Catalytic Activity of Topo II but Not Topo I by Dehydroequol

The stepwise removal of DNA knots (unknotting) requires transient double-strand breakage followed by strand passage and relegation. Type II topoisomerases uniquely catalyse this reaction. The effect of dehydroequol on topo II catalytic activity is displayed in FIG. 1. Unknotted DNA from a mutant bacteriophage (P4 Virl del1O) is used as a reaction substrate that migrates as a smear (due to the variable number of knots). In the presence of topo II, topological DNA knots are removed, and the reaction product (unknotted DNA) migrates as a single band. Dehydroequol inhibited this reaction in a dose-dependent manner as shown in FIG. 1. Complete inhibition was evident at 100 μg/ml dehydroequol. It was determined from densitometric measurements of the unknotted band that 50% inhibition (IC₅₀) was at about 20 μg/ml dehydroequol. The effect of dehydroequol was comparable to that of VP-16, which was used as a positive control.

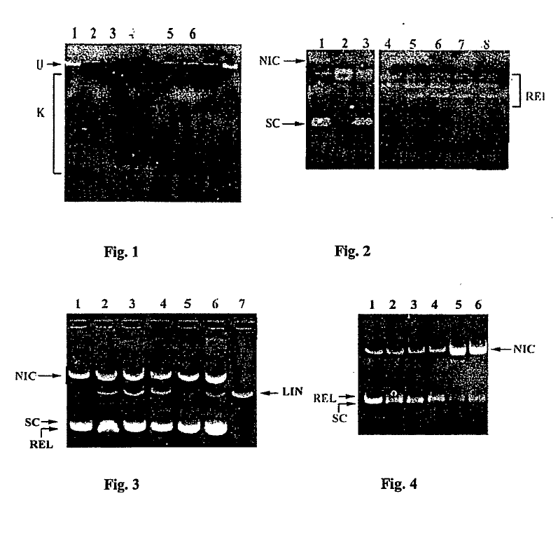

To determine if dehydroequol is a selective inhibitor of topo II, its effect was evaluated in the topo I-mediated relaxation of plasmid DNA in the absence of ATP. Topo II can also relax supercoiled plasmid DNA, but it requires ATP. FIG. 2 shows that purified human topo I relaxes supercoiled plasmid DNA (lane 2). Camptothecin, a known topo I inhibitor, prevents pUC8 DNA relaxation (lane 3), but dehydroequol at concentrations up to 100 μg/ml did not inhibit this topo 1-catalysed reaction (lanes 4-8). These results show that dehydroequol does not inhibit topo I and therefore is a topo II-specific inhibitor.

Induction of Topo I-Mediated Double-Strand DNA Cleavage but not Topo I-Mediated DNA Single-Strand Breakage by Dehydroequol

A linearisation assay was employed to determine if dehydroequol is a topo II poison. Double-strand breakage results in the appearance of linear DNA. Dehydroequol in the presence of topo II, followed by treatment with proteinase K/SDS, effectively produced the linear form of plasmid DNA (FIG. 3, lanes 2-4), indicating that it stabilises the cleavable complex. This effect, which was evident at 10 μg/ml, peaked at 30 μg/ml. In the absence of topo II (lane 1) or proteinase K/SDS (not shown), dehydroequol did not produce linear DNA. The effect of dehydroequol on topo II-mediated DNA strand breakage is unexpectedly much greater than genistein (lane 5) and comparable to VP-16 (lane 6), which were used as positive controls. Since enzyme denaturation and digestion are necessary to release the DNA cleavage, these data demonstrate that dehydroequol-induced DNA breakage is mediated by topo II.

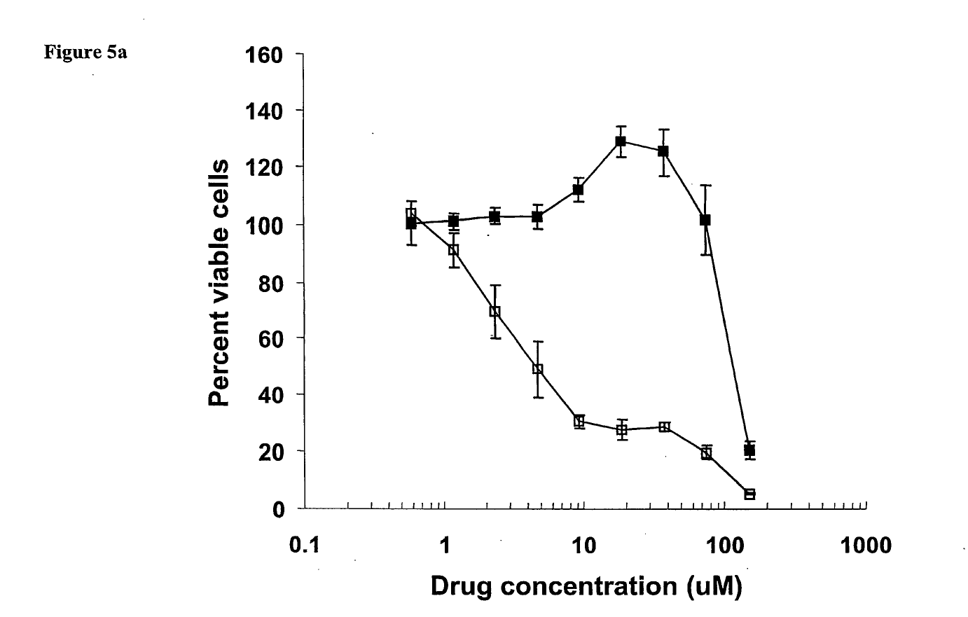

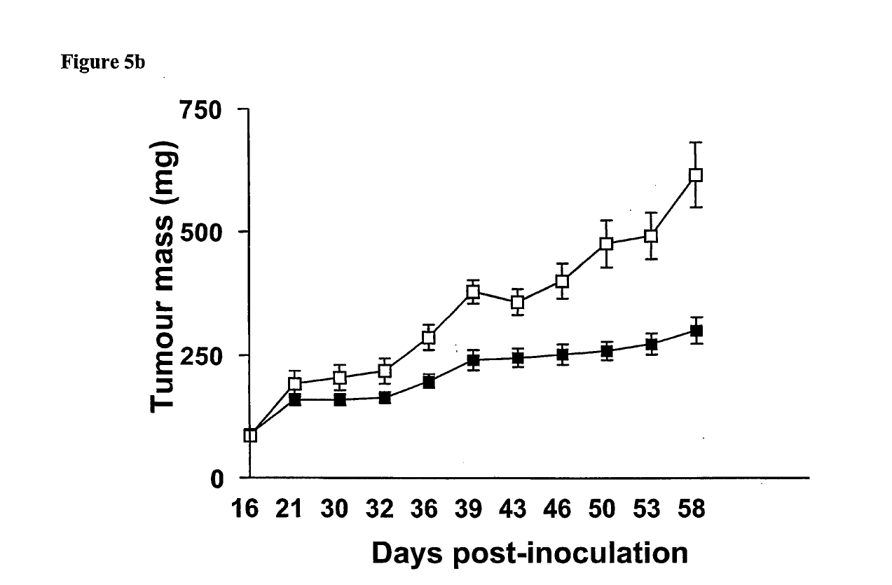

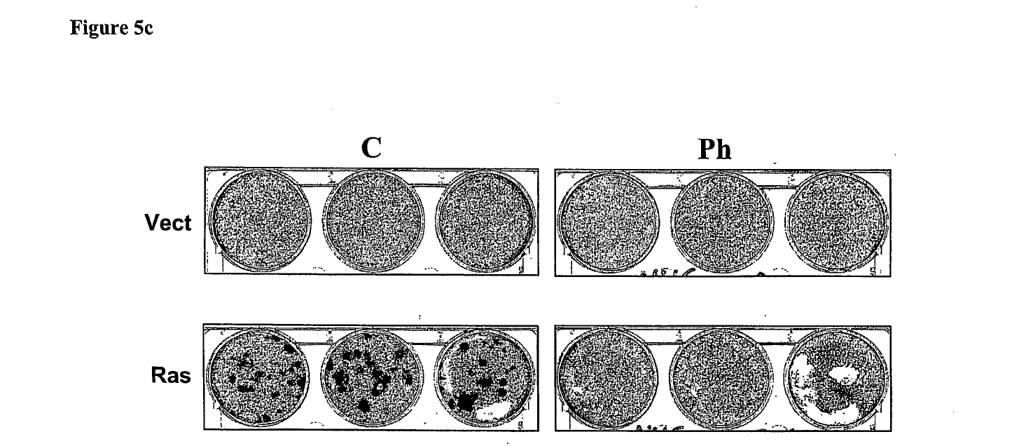

Topo I poisons trap the enzyme-DNA reaction intermediate and, following the digestion of the enzyme, produce single-strand DNA breaks (nicks). Under the electrophoretic conditions that were used in the experiment shown in FIG. 4, covalently closed circular (supercoiled or relaxed) plasmid DNA migrates on the bottom of the gel. In the presence of a topo I poison that stabilises the cleavable complex, and following denaturation and degradation of the enzyme with proteinase K/SDS, the resulting nicked DNA migrates on the top of the gel. Dehydroequol at 20 and 100 μg/ml failed to produce nicked DNA (lanes 3 and 4). Camptothecin, a known topo I poison, produced single-strand DNA cleavage indicated by an increase in the nicked form of DNA, as expected (lanes 5 and 6). These results demonstrate that dehydroequol is devoid of topo I inhibitory effects and therefore is a topo II-specific poison.

Materials

The bacteriophage P4 Vir1 del1O was isolated as described previously [23]. pUC8 DNA was isolated from Escherichia coli by the alkaline lysis method. Reagents, assay buffers, human topo I, human topo II, and pRYG DNA were purchased from Topogen (Columbus, Ohio). Dehydroequol was provided by Novogen (North Ryde, NSW). Genistein was purchased from Indofine Chemical Co. (Somerville, N.J.). All other reagents, chemicals, and drugs were purchased from Sigma Chem. Co. (St. Louis, Mo.). Stock solutions were prepared in DMSO at 20 mg/ml, stored at −20° C., and diluted with distilled water just before the assay.

Topo I-Mediated Plasmid Relaxation Assay

For the determination of topoisomerase (topo) I catalytic activity, pUC8 DNA was used as the substrate in a reaction volume of 20 μl containing the following: 10 mM Tris-HCl, pH 7.9, 1 mM EDTA, 150 mM NaCl, 0.1% BSA, 0.1 mM spermidine, 5% glycerol, and 2 units of purified human topo I. The inhibitor, when applicable, was added as indicated, and the reaction was initiated by the addition of the enzyme. Reactions were carried out at 37° C. for 30 min. Gel electrophoresis was performed at 4 V/cm for 5 h in Tris-borate-EDTA buffer. For the quantitative determination of topo I activity, photographic negatives were scanned. The area representing supercoiled DNA, migrating as a single band at the bottom of the gel, was determined. The concentrations of the inhibitor that prevented 50% of the supercoiled DNA from being converted into relaxed DNA (IC₅₀ values) were determined by averaging the data from at least three experiments.

Topo I-Mediated Plasmid-Nicking Assay

Topo I poisons enhance topo I-mediated pUC8 DNA cleavage under the reaction conditions provided by the supplier of the enzyme (Topogen, Inc.). Briefly, 20 μl of reaction mixtures contained 10 mM Tris-HCl, pH 7.5, 1 mM EDTA, 100 mM NaCl, 1 μl of the test agent (or solvent), 0.5 μg of pUC8, and 10 units of human topo I (added last). After a 30-min incubation at 37° C., SDS-proteinase K was added, and, following a 30-min incubation at 37° C., samples were extracted with CHCl₃-isopropanol and electrophoresed on a 1% agarose gel containing 0.5 μg/ml ethidium bromide. Gels were photographed, and photographic negatives were scanned.

Topo II-Mediated P4 Unknotting Assay

To determine topoisomerase (topo) II catalytic activity, knotted DNA that had been isolated from the tailless capsids of the bacteriophage P4 Virl del1O was used as the substrate. Reaction mixtures contained 50 mM Tris-HCl, pH 8.0, 120 mM KCl, 10 mM MgCl₂, 0.5 mM ATP, and 0.5 mM dithiothreitol. The topo II inhibitor was added prior to the addition of 2 units of human topo II. Reactions (20 μl final volume) were initiated by adding 0.6 μg of knotted DNA and carried out at 37° C. for 30 min. Reactions were terminated by the addition of 5 ml of a stop solution containing 5% SDS, 50 mM EDTA, 25% ficoll, and 0.05 mg/ml bromophenol blue. Samples were loaded on 0.8% agarose gels, and electrophoresis was performed at 4 V/cm for 5 h in Tris-borate-EDTA buffer. Gels were stained with ethidium bromide, destained, and photographed over a UV light source. For the quantitative determination of topo II activity, photographic negatives were densitometrically scanned. Unknotted DNA, migrating as a single band at the top of the gel, was measured in this manner. The concentration of the inhibitor preventing 50% of the substrate (knotted DNA) from being converted into the reaction product (unknotted DNA) was determined from a standard curve. By averaging three to four such experiments, the IC₅₀ values were determined.

Topo II-Mediated Plasmid Linearisation Assay

Topo II poisons enhance topo II-mediated DNA cleavage and can be identified with the linearisation assay under the reaction conditions provided by the supplier of the enzyme (Topogen, Inc.). Briefly, 20-μl reaction mixtures contained 30 mM Tris-HCl, pH 7.6, 3 mM ATP, 15 mM β-mercaptoethanol, 8 mM MgCl₂, 60 mM NaCl, 1 μl of the test agent (or solvent), 0.3 μg of pRYG, and 10 units of human topo II (added last). After a 15-min incubation at 37° C., SDS-proteinase K was added, and following a 15 min incubation at 37° C., samples were extracted with CHCl₃-isopropanol and electrophoresis was performed on a 1% agarose gel containing ethidium bromide. Gels were photographed, and photographic negatives were scanned.

### EXAMPLE 2

tNOX Inhibition, Tumour Cell Cycle Arrest and Apoptosis

A 96-well plate assay was used containing human cervical carcinoma (HeLa) in a dithiodipyridine substrate. Dehydroequol present in a concentration of 10 μm was shown to inhibit tNOX activity leading to HeLa cell apoptosis.

Subsequent clinical studies are shown to demonstrate the therapeutic benefits and activities of the compounds of formulae I and II.

### EXAMPLE 3

Potent Anti-Tumour and Anti-Angiogenic Properties of Phenoxydiol (Dehydroequol)

This example describes the potent anti-tumour/anti-cancer, anti-angiogenic activity, and anti-inflammatory activity of dehydroequol as a representative compound of the formula II.

Methods

Cells: Human umbilical vein endothelial cells (HUVEC) were prepared and grown as previously described³⁰. The cells were used between passages 2 and 6 for all experiments. The human prostatic adenocarcinoma cell line, LNCaP was obtained from American Tissue Type Collection and maintained according to the instructions of the supplier.

Assays:

Proliferation assay: HUVEC were seeded at 3×10³ cells per gelatin-coated microtitre well and grown in complete medium for 3 days. Dehydroequol (De) was added 3 hours after plating. Proliferation was measured using the MTT assay (Promega, Wis., USA) and is given as the rate of proliferation over the 3 days±SEM of quadruplicate determinations for each group. Proliferation of LNCaP cells was determined using the MTT assay. Cells were seeded at 2.5×10³ cells per microtitre well. De was added 4 days after plating and assayed after a further 5 days of growth. Cell viability is expressed as a percentage of control untreated cells.

Migration assay: HUVEC were plated at 5×10⁵ cells per gelatin-coated 6 well dish and grown to confluence over 48 hours. Wounds were made in the monolayer, cells washed 3 times and fresh complete media with or without De (10 μg/ml final concentration) added. Migration was viewed over the following 18-72 hours.

In vitro tube assay: Capillary tube formation in a collagen gel was performed essentially as described by Gamble et al^{30,31}. Tubes were formed in the presence of the tumour promoter phorbol myristate acetate (PMA) and the anti β₁ integrin antibody RMACII. De (10 μg/ml final) was added at the time of plating the cells onto the gel.

Athymic mice xenograft assay: LNCaP human prostate cells were implanted subcutaneously into athymic Balb/c mice and De (2 mg uid) administered orally from the time of cell inoculation for 5 days per week. Animals were killed 58 days post-implantation and tumour mass (mg) calculated from the formula (width²×length)/2 as used in NCI.

Sphingosine Kinase activity assay: SK activity was measured in vitro by incubating the cytosolic fraction with 10 μM sphingosine-BSA complex and [γ³²P]ATP (1 mM, 0.5 mCi/ml) for 15 minutes at 37° C., as previously described⁴². Stimulation was with TNFα (1 ng/ml) (rhTNF-α; R & D Systems, Minneapolis Minn. USA), PMA (100 ng/ml) and IL-1β (100 units/ml) (hrIL-1β; Immunex, Seattle Wa. USA) for 10 minutes. Cells were treated with De or DMSO vehicle as control for 18 hours prior to stimulation.

Focus formation assay: Low passage NIH 3T3 cells were transfected with either human ras gene (Ras) or empty vector (Vect) control¹⁹. Two days later the transfected cells were seeded in 6 well plates. After reaching confluence, the cells were cultured in the vehicle DMSO or De (10 μg/ml final) for 3 weeks with media change every 3-4 days (±De). Foci were scored after staining with 0.5% crystal violet. One experiment of two performed is shown.

Northern blot analysis: HUVEC were plated at 1×10⁶ cells per 25 cm² flask and grown for 48 hours. Following treatment with DMSO vehicle or De (10 μg/ml final) for 18 hours, total RNA was harvested and purified using TRIZOL reagent (Invitrogen—Life Technologies, Groningen, Netherlands) according to the manufacturer's protocol. Northern blot analysis was performed using 8 μg total RNA transferred onto Hybond-N membranes (Amersham Biosciences, Buckinghamshire, England) and probed with human MMP-2 and GAPDH cDNAs using a Strip-EZ PCR stripAble PCR Probe synthesis and removal Kit (Ambion, Tex., USA).

E selectin and VCAM-1 assays: HUVEC were plated at 5×10⁵ cells per 6 well dish. Cells were treated with DMSO vehicle or De (10 μg/ml final) for 18 hours prior to TNFα (1 ng/ml) addition. After 4 or 18 hours, cells were washed with PBS and incubated with antibodies against E selectin (Mab49-1B11) or VCAM-1 (Nab51-10C9) for 30 min. Goat F(ab′)2 fragment mouse-IgG(H+L)-FITC antibody (Immunotech, Marseille, France) was then added for 30 min. The cells were then trypsinised and FACS analysed on a Coulter Epics® XL-MCL (Beckman Coulter). Results are expressed as arbitrary units relating to the mean intensity of FITC fluorescence.

IL8 assay: IL-8 assays were performed using a “Quantikine Human IL-8 Immunoassay” (R & D Systems, Minneapolis Minn. USA).

Results

Dehydroequol (“De”) showed anti-proliferative effects on the leukaemic cell lines K562 and HL60 (IC₅₀ of 3.0 and 1.5 μg/ml respectively), breast cancer line MCF7 (1.5 μg/ml IC₅₀), colon cancer lines HT29 and CaCo-2 (IC₅₀ of 15.0 and 1.0 μg/ml respectively), and prostate cancer lines DU145, PC3 and LNCaP (IC₅₀ of 3.0, 2.0 and 1.5 μg/ml respectively). De is 5-20 times more potent as a cytotoxic agent than genistein on these cell lines. The comparative data of De and genistein on LNCaP is shown in FIG. 1a. In vitro cytotoxicity assessment of De against LnCaP showed an IC₅₀ (μM) of 4.4 (n=3 separate experiments). Xenograft assays measuring the growth of the prostate cancer line LNCaP demonstrated that De was effective at inhibiting the tumour growth (49% tumour growth inhibition p<0.0003; De treated versus control group) (FIG. 5b).

The Ras oncogene has been linked to the development of many human cancers by either over-expression or mutations in the normal gene^{28,29}. De treatment of Ras transformed NIH 3T3 cells completely inhibited the development of colonies although still maintaining the viability of normal NIH 3T3 cells (FIG. 5c). This suggests that De specifically targets the highly proliferative Ras transformed cells with little or no effect against normal cells.

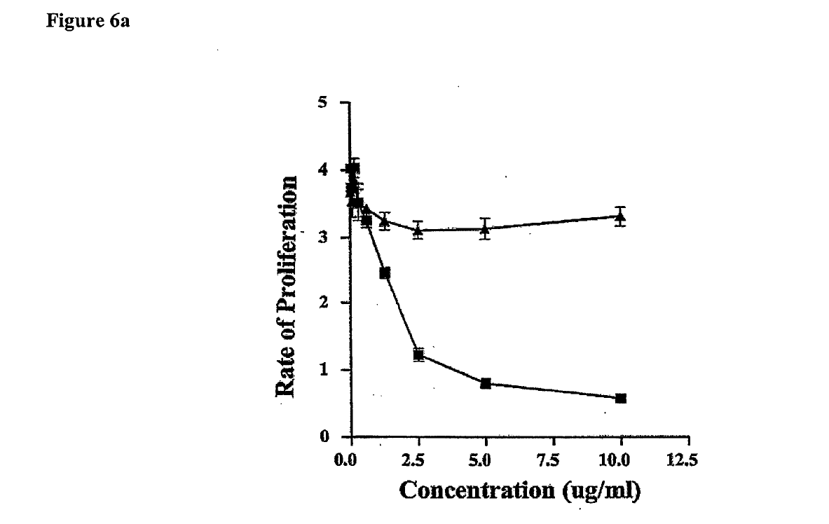

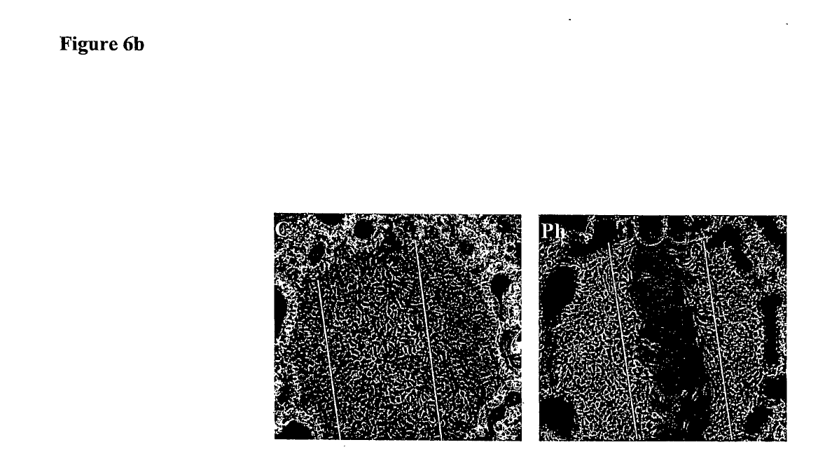

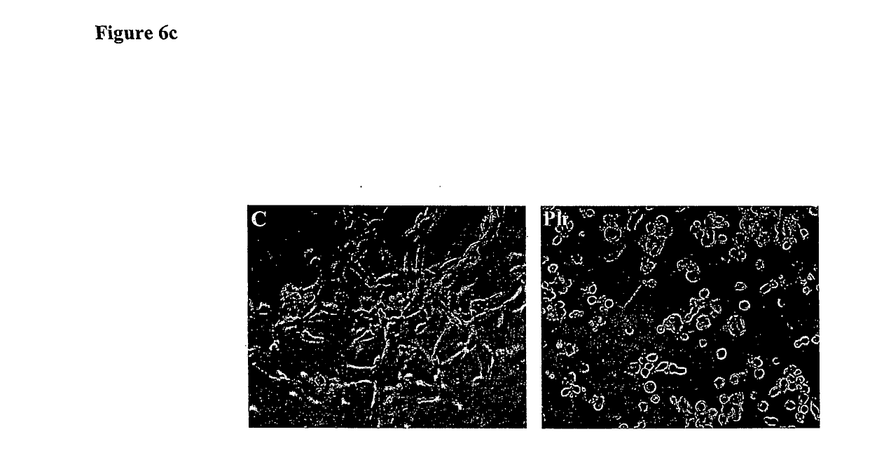

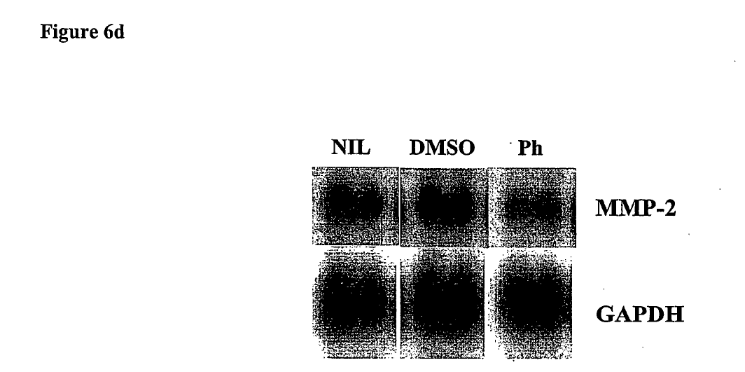

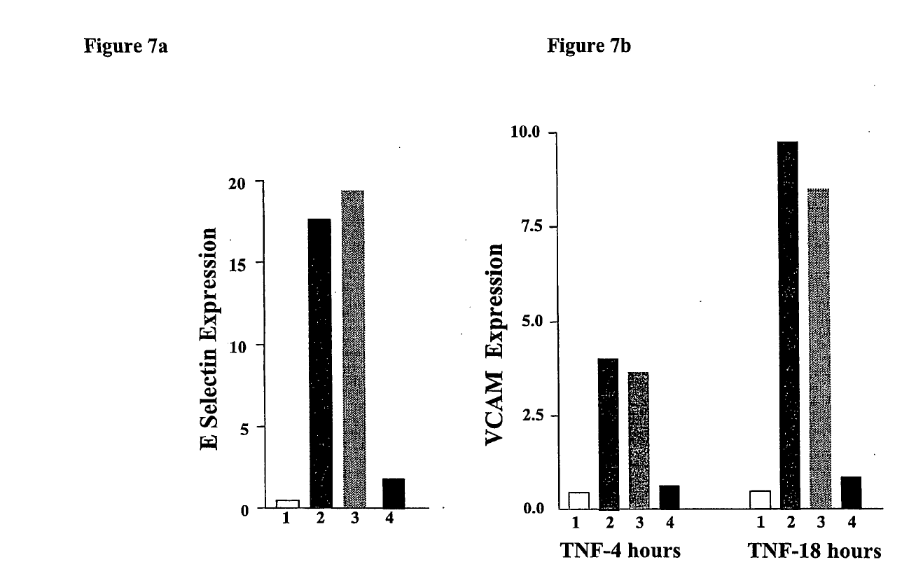

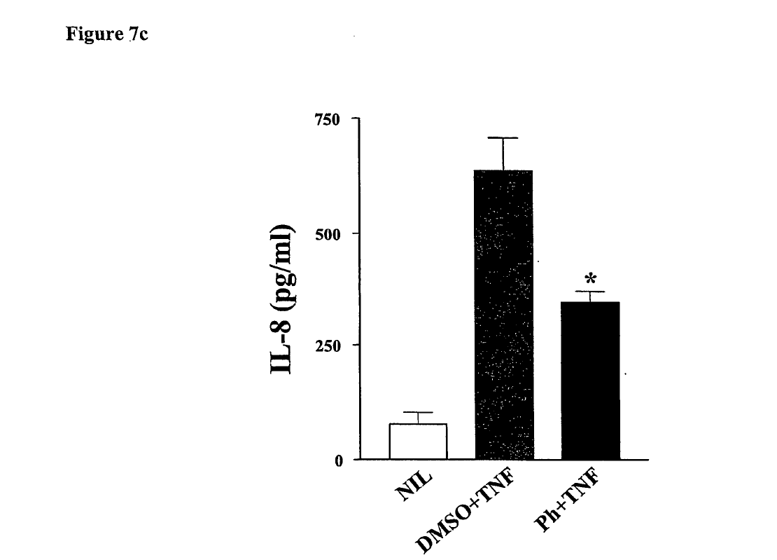

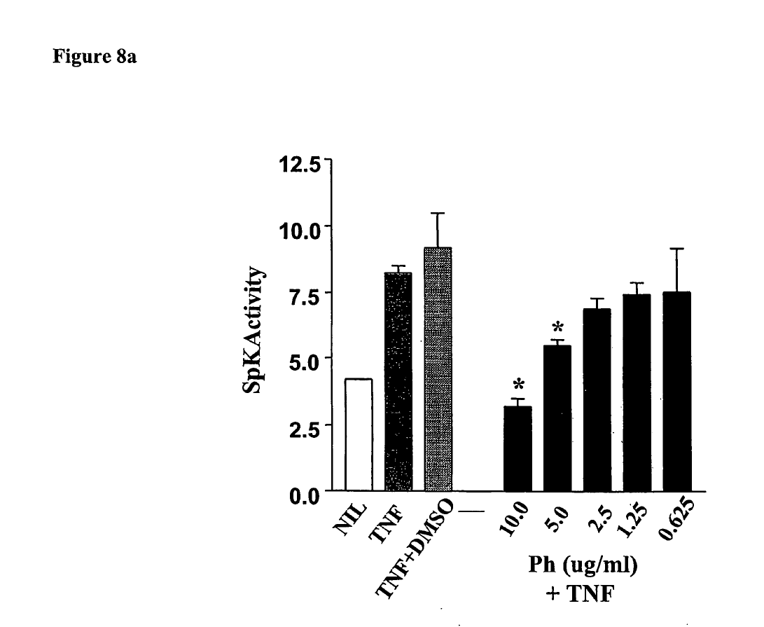

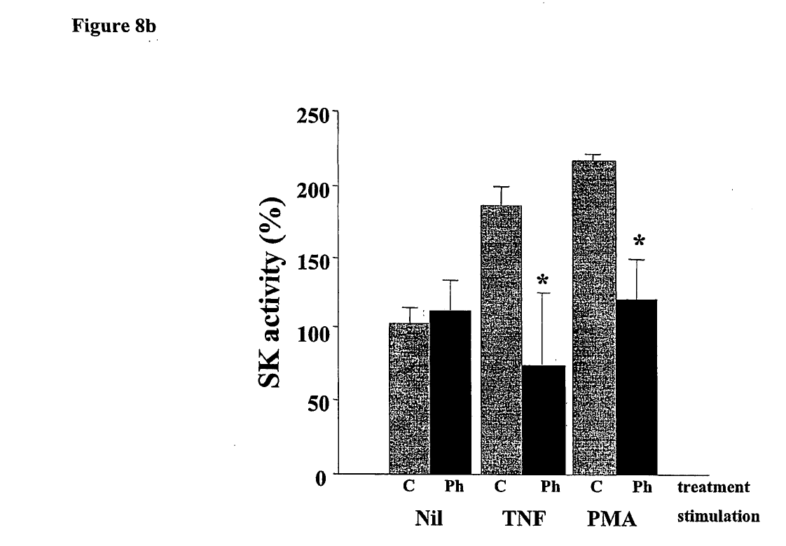

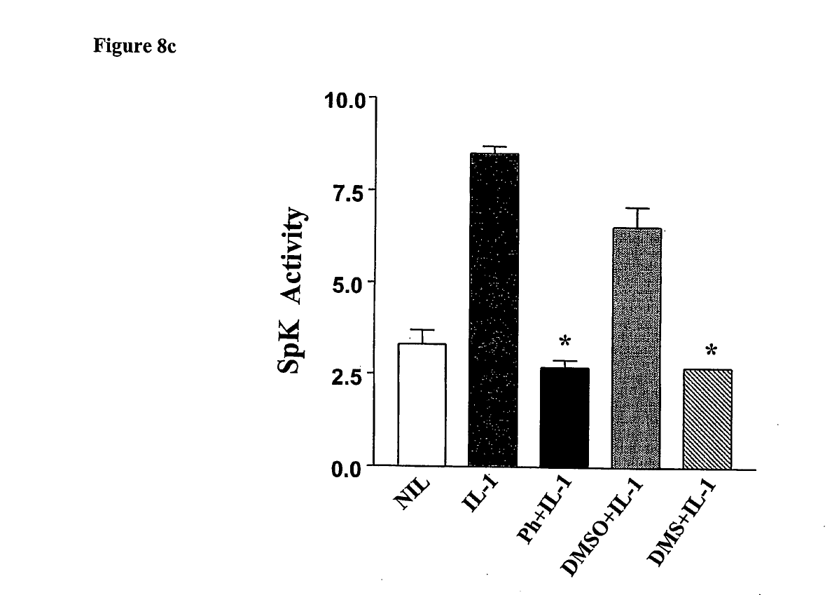

Solid tumour growth is not only dependent on the ability of the transformed cells to evade normal mechanisms that control cell survival and proliferation but also on the ability of the cells to stimulate the expansion of the vascular compartment through angiogenesis. To determine whether De has anti-angiogenic activity in addition to the anti-tumourgenic activity, De was tested in endothelial cell (EC) proliferation, migration and capillary tube formation assays, in vitro hallmarks of angiogenesis. The results demonstrate that De inhibits all these aspects of EC function (FIGS. 6a, b and c). Results presented are of single experiments, however, De was consistent in its inhibitory effect over multiple experiments performed. For example, 10 μg/ml De showed a 91%±3% inhibition of proliferation in five experiments performed. The results also demonstrate that De is not cytotoxic for normal EC since, in the migration assays, EC not in the vicinity of the wound remained viable even in the presence of De for 72 hours. Further, in proliferation assays, De inhibited the proliferative potential of the cells but did not cause cell death.

In the collagen gel assay, the cells treated with vehicle control showed the typical large capillary tubes forming at different plains throughout the gel similar to that which we have reported previously,^{30,31}. However, morphological assessment of the cells treated with De suggested that De may inhibit the invasion of the cells into the gel since the cells remained rounded and were seen on top of the gel even after many hours. Invasion of EC into the gel as well as neovascularisation in vivo is dependent on the synthesis and secretion of the matrix degrading enzymes, such as the metalloproteases^{32,32}. The matrix metalloproteinase, MMP-2 is essential in the “angiogenic switch” and inhibition of its activity prevents angiogenesis³⁴. Thus, MMP-2 was targeted for investigation. Northern blot analysis of endothelial cells treated with 10 μg/ml of De for 18 hours showed an inhibition in the level of RNA for MMP-2 (FIG. 6d). In the two experiments performed, there was a 68.3% and 46.7% decrease in level of mRNA for MMP-2.

Angiogenesis is normally associated with inflammation and both now appear coordinately regulated^{35,36}. Inflammation is exemplified by expression of adhesion molecules on the endothelium and secretion of the chemotactic cytokines which are responsible for egress of inflammatory cells from the circulation. Inflammatory cells such as neutrophils, lymphocytes and monocytes are sources of many of the potent angiogenic factors such as vascular endothelial cell growth factor³⁵. De inhibits the induction of the adhesion molecules E selectin and VCAM-1 to both TNF and IL-1 on the endothelium and also inhibits the secretion of EL-8 (FIGS. 7a and b). However, De did not display a general inhibitory effect on the EC since there were no changes seen in the level of expression of PECAM-1 or VEGF receptor 2 as determined by flow cytometric analysis (data not shown) after De treatment. Thus, De inhibits both the angiogenic process per se and also the inflammatory component which amplifies the angiogenic state.

The highly conserved lipid kinase, Sphingosine Kinase (SK), which phosphorylates sphingosine yielding sphingosine-1-phosphate has been implicated in the promotion of cell survival, growth and transformation³⁷⁻⁴⁰ and in the function of oncogenes such as Ras⁴¹. SK is also a key mediator in the regulation of EC activation and proliferation⁴²⁻⁴⁴. Sphingosine-1-phosphate is also involved in the angiogenic process⁴⁵⁻⁴⁷ and has recently been shown to be involved in the VEGF signalling pathway⁴⁸. To test whether De could exert its action at least potentially through inhibition of the SK pathway, the SK activity generated in the presence or absence of De was assessed. The results show (FIG. 4) that in a dose-dependent manner, De inhibits SK generated by the stimulation of EC with TNF (a), the tumour promoter, phorbol myristate acetate (PMA) (b) and IL-1 (c). De is equipotent in its inhibition with the inhibitor of SK, N,N-dimethylsphingosine (DMS). De has no effect on the basal levels of SK activity, suggesting a specific effect on the activation phase of the enzyme. De does not affect the TNF-stimulated sphingomyelinase activity (data not shown) suggesting De does not affect the sphingomyelin metabolic pathway.

This example describes the powerful anti-tumour and anti-angiogenic effects of dehydroequol (De). The most striking observation is the potency of dehydroequol in a range of assays measuring endothelial functions with relevance to angiogenesis. These include endothelial migration and expression of enzymes required for matrix breakdown, proliferation, expression of adhesion molecules, and in vitro tube formation. Equally striking was the lack of toxicity, at the doses used, of dehydroequol for resting endothelial cells and for the untransformed 3T3 cells. These properties of the drug might predict potent anti-tumour effects but with limited general toxicity, and indeed, these have been observed in these studies.

The mechanisms underlying the diverse actions of this drug are not fully elucidated. However, it is a potent (direct or indirect) inhibitor of at least three relevant enzyme systems⁴⁹. The first two of these, protein tyrosine kinases and topoisomerases, have been implicated in cellular activation and proliferation for a long time^{13,15,49}. We now report here the inhibition of a third enzyme system, the lipid kinase sphingosine kinase, which has recently been implicated in endothelial activation and angiogenesis as well as oncogenesis. We note that the major action of De is on the inhibition of SK activation by agents like TNF and IL-1 while showing little effect on the basal activity of SK. This result could account for the selectivity of the agent, showing dramatic inhibition on transformed tumour cells and on EC involved in the process of angiogenesis while having little or no effect on the viability of normal cells such as NIH 3T3 cells or EC. Thus, De may target specifically the activation phase of SK, such as what would take place during Ras induced transformation or in the activation of EC necessary for angiogenesis. These results highlight the prospect that De may be an effective and safe anti-cancer agent.

Although anti-angiogenic therapy for cancer carries with it a lot of excitement, the results of many trials have been disappointing, suggesting that anti-angiogenic therapy should be combined with other anti-cancer modalities. For this reason, it is of great interest to note that De has direct inhibitory effects on the growth of several types of cancer cells in vitro and in vivo thus showing that in one drug the anti-angiogenic and anti-cancer properties are combined.

The invention has been described herein, with reference to certain preferred embodiments, in order to enable the reader to practice the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications cited herein are hereby incorporated by reference.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification individually or collectively, and any and all combinations of any two or more of said steps or features.

The reference to any prior art in this specification is not, and should not be taken as, an acknowledgment or any form of suggestion that that prior art forms part of the common general knowledge in the field of endeavour.

### ENDNOTE REFERENCES

1. Joannou, G E, Kelly, G E, Reeder, A Y, Waring, M, Nelson, C. A Urinary profile Study of Dietary Phytoestrogens. The Identification and Mode of Metabolism of New Isoflavonoids. Journal of Steroid Biochemistry and Molecular Biology, 1995, 54, 176-184.
2. Miyase, T, Sano, M. Antioxidant from Lespedeza holoba. Phytochemistry 1999, 52(2), 303-310.
3. Kellner, U, Rudolph P, Parwaresch R. Human DNA-Topoisomerases—Diagnostic and Therapeutic Implications for Cancer. Onkologie 2000, 23, 424-430.
4. Liu, L F. DNA topoisomerase poisons as antitumor drugs. Annu Rev Biochem 1989, 58, 351-75.
5. Heck, M M, Earnshaw W C. Topoisomerase II: A specific marker for cell proliferation. J Cell Biol 1986, 103, 2569-81.
6. Heck, M M, Hittelman W N, Earnshaw W C. Differential expression of DNA topoisomerases I and II during the eukaryotic cell cycle. Proc Natl Acad Sci USA 1988, 85, 1086-90.
7. Davis, J N, Singh B, Bhuiyan M, Sarkar F H. Genistein-induced upregulation of p21WAF1, downregulation of cyclin B, and induction of apoptosis in prostate cancer cells. Nutr Cancer 1998, 32, 123-31.
8. Deffie, A M, Bosman D J, Goldenberg G J. Evidence for a mutant allele of the gene for DNA topoisomerase II in adriamycin-resistant P388 murine leukemia cells. Cancer Res 1989, 49, 6879-82.
9. Constantinou, A, Kiguchi K, Huberman E. Induction of differentiation and DNA strand breakage in human HL-60 and K-562 leukemia cells by genistein. Cancer Res 1990, 50, 2618-24.
10. Kiguchi, K, Constantinou A I, Huberman E. Genistein-induced cell differentiation and protein-linked DNA strand breakage in human melanoma cells. Cancer Commun 1990,2, 271-7.
11. Okura, A, Arakawa H, Oka H, Yöshinari T, Monden Y. Effect of genistein on topoisomerase activity and on the growth of [Val 12]Ha-ras-transformed NIH 3T3 cells. Biochem Biophys Res Commun 1988, 157, 183-9.
12. Constantinou, A, Mehta R, Runyan C, Rao K, Vaughan A, Moon R. Flavonoids as DNA topoisomerase antagonists and poisons: structure-activity relationships. J Nat Prod 1995, 58, 217-25.
13. Markovits, J, Linassier C, Fosse P, Couprie J, Pierre J, Jacquemin-Sablon A, Saucier J M, Le Pecq J B, Larsen A K. Inhibitory effects of the tyrosine kinase inhibitor genistein on mammalian DNA topoisomerase II. Cancer Res 1989, 49, 5111-7.
14. Uckun, F M, Messinger Y, Chen C L, O'Neill K, Myers D E, Goldman F, Hurvitz C, Casper J T, Levine A. Treatment of therapy-refractory B-lineage acute lymphoblastic leukemia with an apoptosis-inducing CD19-directed tyrosine kinase inhibitor. Clin Cancer Res 1999, 5, 3906-13.
15. Strick, R, Strissel P L, Borgers S, Smith S L, Rowley J D. Dietary bioflavonoids induce cleavage in the MLL gene and may contribute to infant leukemia. Proc Natl Acad Sci USA 2000, 97, 4790-5.
16. Burden, D A, Osheroff N. Mechanism of action of eukaryotic topoisomerase II and drugs targeted to the enzyme. Biochim Biophys Acta 1998, 1400, 139-54.
17. Hansen, H H. Management of small-cell cancer of the lung. Lancet 1992, 339, 846-9.
18. Beere, E M, Chresta C M, Alejo-Herberg A, Skladanowski A, Dive C, Larsen A K, Hickman J A. Investigation of the mechanism of higher order chromatin fragmentation observed in drug-induced apoptosis. Mol Pharmacol 1995, 47, 986-96.
19. Snapka, R M, Kwok K, Bernard J A, Harling O K, Varshavsky A. Post-separation detection of nucleic acids and proteins by neutron activation. Proc Natl Acad Sci USA 1986, 83, 8939-42.
20. Fortune, J M, Osheroff N. Topoisomerase II as a target for anticancer drugs: when enzymes stop being nice. Prog Nucleic Acid Res Mol Biol 2000, 64, 221-53.
21. Jensen, P B, Sehested M. DNA topoisomerase II rescue by catalytic inhibitors: a new strategy to improve the antitumor selectivity of etoposide. Biochem Pharmacol 1997, 54, 755-9.
22. Meng, L H, Zhang J S, Ding J. Salvicine, a novel DNA topoisomerase II inhibitor, exerting its effects by trapping enzyme-DNA cleavage complexes. Biochem Pharmacol 2001, 62, 733-41.
23. Liu, L F, Davis J L, Calendar R. Novel topologically knotted DNA from bacteriophage P4 capsids: studies with DNA topoisomerases. Nucleic Acids Res 1981, 9, 3979-89.
24. Morré et al (2002) Biochemistry, Vol. 41 No. 40, pages 11941-11945.
25. Morré, D. J. (1995) Biochim. Biophys. Acta. 1240, 201-208.
26. Morré, D. J. (1998) in Plasma Membrane Redox Systems and Their Role in Biological Stress and Disease (Asard et al editors), pp 121-156, Kluwer Academic Publishers, Dordrecht, the Netherlands.
27. Wang et al (2001) Biochem. Biophys. Acta. 1539, 192-204
28. Przybojewska, B., Jagiello, A. & Jalmuzna, P. H-RAS, K-RAS, and N-RAS gene activation in human bladder cancers. Cancer Genet. Cytogenet. 121, 73-77 (2000).
29. Bos, J. L. ras oncogenes in human cancer: a review. Cancer Res. 49, 4682-4689 (1989).
30. Gamble, J. R. et al. Regulation of in vitro capillary tube formation by anti-integrin antibodies. J. Cell Biol. 121, 931-943 (1993).
31. Gamble, J. et al. B1 integrin activation inhibits in vitro tube formation: effects on cell migration, vacuole coalescence and lumen formation. Endothelium 7, 23-34 (1999).
32. Naglich, J. G. et al. Inhibition of angiogenesis and metastasis in two murine models by the matrix metalloproteinase inhibitor, BMS-275291. Cancer Res. 61, 8480-8485 (2001).
33. Haas, T. L., Davis, S. J. & Madri, J. A. Three-dimensional type I collagen lattices induce coordinate expression of matrix metalloproteinases MT1-MMP and MMP-2 in microvascular endothelial cells. J Biol Chem 273, 3604-3610 (1998).
34. Fang, J. et al. Matrix metalloproteinase-2 is required for the switch to the angiogenic phenotype in a tumor model. Proc Natl Acad Sci U S A 97, 3884-3889 (2000).
35. Salven, P., Hattori, K., Heissig, B. & Rafii, S. Interleukin-1 alpha promotes angiogenesis in vivo via VEGFR-2 pathway by inducing inflammatory cell VEGF synthesis and secretion. FASEB J 16, 1471-1473 (2002).
36. Klein, S. et al Alpha 5 beta 1 integrin activates an NF-kappa B-dependent program of gene expression important for angiogenesis and inflammation. Mol Cell Biol 22, 5912-5922 (2002).
37. Olivera, A. & Spiegel, S. Sphingosine-1-phosphate as second messenger in cell proliferation induced by PDGF and FCS mitogens. Nature 365, 557-560 (1993).
38. Van Brocklyn, J. R. et al. Dual actions of sphingosine-1-phosphate: extracellular through the Gi-coupled receptor Edg-1 and intracellular to regulate proliferation and survival. J Cell Biol. 142, 229-240 (1998).
39. Olivera, A. et al. Sphingosine kinase expression increases intracellular sphingosine-1-phosphate and promotes cell growth and survival [In Process Citation]. J Cell Biol. 147, 545-558 (1999).
40. Hong, G., Baudhuin, L. M. & Xu, Y. Sphingosine-1-phosphate modulates growth and adhesion of ovarian cancer cells. FEBS Lett. 460, 513-518 (1999).
41. Xia, P. et al. An oncogenic role of sphingosine kinase. Curr. Biol 10, 1527-1530 (2000).
42. Xia, P. et al. Tumor necrosis factor-alpha induces adhesion molecule expression through the sphingosine kinase pathway. Proc Natl Acad Sci U S. A 95, 14196-14201 (1998).
43. Xia, P., Wang, L., Gamble, J. R. & Vadas, M. A. Activation of sphingosine kinase by tumor necrosis factor-alpha inhibits apoptosis in human endothelial cells. J Biol. Chem. 274, 34499-34505 (1999).
44. Xia, P., Vadas, M. A., Rye, K. A., Barter, P. J. & Gamble, J. R. High density lipoproteins (HDL) interrupt the sphingosine kinase signaling pathway. A possible mechanism for protection against atherosclerosis by HDL. J Biol. Chem. 274, 33143-33147 (1999).
45. Ancellin, N. et al. Extracellular export of sphingosine kinase-1 enzyme. Sphingosine 1-phosphate generation and the induction of angiogenic vascular maturation. J Biol Chem 277, 6667-6675 (2002).
46. English, D. et al. Induction of endothelial cell chemotaxis by sphingosine 1-phosphate and stabilization of endothelial monolayer barrier function by lysophosphatidic acid, potential mediators of hematopoietic angiogenesis. J Hematother. Stein Cell Res. 8, 627-634 (1999).
47. Lee, O. H. et al. Sphingosine 1-phosphate induces angiogenesis: its angiogenic action and signaling mechanism in human umbilical vein endothelial cells. Biochem Biophys. Res. Commun. 264, 743-750 (1999).
48. Shu, X., Wu, W., Mosteller, R. D. & Broek, D. Sphingosine kinase mediates vascular endothelial growth factor-induced activation of ras and mitogen-activated protein kinases. Mol Cell Biol 22, 7758-7768 (2002).
49. Constantinou, A. I. & Husband, A. Phenoxodiol (2H-1-Benzopyran-7-0,1,3-4-hydroxyphenyl), a Novel Isoflavone Derivative, Inhibits DNA Topoisomerase II by Stabilizing the Cleavable Complex. Anticancer Research (in press).

1. A method for the treatment, prevention or amelioration of diseases associated with aberrant cell survival, aberrant cell proliferation, abnormal cellular migration, abnormal angiogenesis, abnormal estrogen/androgen balance, dysfunctional or abnormal steroid genesis, degeneration including degenerative changes within blood vessel walls, inflammation, or immunological imbalance, which comprises administering to a subject a compound of the formula II (as herein defined) or a pharmaceutically acceptable salt or derivative thereof.

2. Use of compounds of the formula II including pharmaceutically acceptable salts and derivatives thereof in the manufacture of a medicament for the treatment, prevention or amelioration of diseases associated with aberrant cell survival, aberrant cell proliferation, abnormal cellular migration, abnormal angiogenesis, abnormal estrogen/androgen balance, dysfunctional or abnormal steroid genesis, degeneration including degenerative changes within blood vessel walls, inflammation or immunological imbalance.

3. A method of inducing apoptosis in cells expressing abnormal cell survival phenotype which comprises contacting said cells with a compound of the formula II including pharmaceutically acceptable, salts thereof optionally in association with a carrier or excipient.

4. A method for inhibiting migration of cells having an abnormal cellular migration phenotype which comprises contacting said cells with a compound of the formula II including pharmaceutically acceptable salts thereof optionally in association with a carrier or excipient.

5. A method for inhibiting angiogenesis in tissue expressing aberrant angiogenic phenotype which comprises contacting said tissue with a compound of the formula II including pharmaceutically acceptable salts thereof optionally in association with a carrier or excipient.

6. A method for the inhibition of topoisomerase II in a mammal which method comprises the step of administering to the mammal a therapeutically effective amount of a compound of formula II or a pharmaceutically acceptable salt or derivative thereof.

7. A method for the treatment, prevention or amelioration of cancer in a mammal which method comprises the step of bringing a compound of formula II or a pharmaceutically acceptable salt or derivative thereof into contact with cancerous tissue in a mammal that is suffering from a tumour, such that neoplastic development in said cancerous tissue is retarded or arrested.

8. A method of claim 7, wherein the neoplastic development is retarded or arrested by the compound of formula II stabilising a cleavable complex of DNA topoisomerase II.

9. A method of inducing apoptosis in cells expressing DNA topoisomerase II which comprises contacting said cells with one or more compounds of the formula II optionally in association with a carrier or excipient.

10. A method of inhibiting DNA topoisomerase II by contacting a DNA topoisomerase cleavable complex with a compound of formula II or a pharmaceutically acceptable salt or derivative thereof to stabilise the cleavable complex.

11. Use of a compound of formula II or a pharmaceutically acceptable salt or derivative thereof in the manufacture of a medicament for the treatment of cancer in a mammal.

12. Use of a compound of formula II or a pharmaceutically acceptable salt or derivative thereof as a DNA topoisomerase II poison.

13. A pharmaceutical composition for the treatment of cancer comprising a compound of formula II or a pharmaceutically acceptable salt or derivative thereof in association with a pharmaceutically acceptable carrier and/or diluent.

14. A synergistic pharmaceutical composition comprising a compound of formula II in admixture with another chemotherapeutic active agent, preferably another topo II poison.

15. A kit comprising a compound of formula II and another chemotherapeutic active agent, preferably another topo II poison.

16. A method according to any one of claims 1 and 3-10 or a use according to any one of claims 2, 11 and 12 wherein the compound of the formula II is dehydroequol.

17. A pharmaceutical composition according to claim 13 or 14 or a kit according to claim 15 wherein the compound of the formula II is dehydroequol.

18. A method for the treatment, prevention or amelioration of cancer in a mammal which comprises the step of bringing a compound of the formula II or a pharmaceutically acceptable salt or derivative thereof into contact with a cancerous tissue in a mammal that is suffering from a tumour, wherein in compounds of the formula II inhibit tNOX associated with said cancerous tissue, such that neoplastic development in said cancerous tissue is retarded or arrested.

19. Use of a compound of the formula II or a pharmaceutically acceptable salt or derivative thereof as a tNOX inhibitor.

20. Use of a compound of the formula II in the manufacture of a medicament for the inhibition of tNOX associated with tumour cells.

21. A pharmaceutical composition comprising a compound of the formula (II) in association with one or more other pharmaceutically active agents.
